(19) United States
(12) Patent Application Publication
(10) Pub. No.: US 2021/0346942 A1
(43) Pub. Date: Nov. 11, 2021

(54) A MOLDING COMPRISING A ZEOLITIC MATERIAL HAVING FRAMEWORK TYPE MFI

(71) Applicant: BASF SE, Ludwigshafen am Rhein (DE)

(72) Inventors: Andrei-Nicolae PARVULESCU, Ludwigshafen am Rhein (DE); Hans-Juergen LUETZEL, Boehl-lggelheim (DE); Ulrich MUELLER, Ludwigshafen am Rhein (DE); Dominic RIEDEL, Ludwigshafen am Rhein (DE); Joaquim Henrique TELES, Ludwigshafen am Rhein (DE); Markus WEBER, Ludwigshafen am Rhein (DE)

(21) Appl. No.: 17/283,975

(22) PCT Filed: Oct. 9, 2019
(86) PCT No.: PCT/EP2019/077372

§ 371 Date: Apr. 9, 2021

(30) Foreign Application Priority Data
Oct. 9, 2018 (EP) 18199428.6

(51) Int. Cl. B22C 1/18; B01J 29/70; B01J 35/10; B01J 6/00; C01B 39/46; B01J 21/08

(52) U.S. Cl. CPC B22C 1/181; B01J 29/70; B01J 21/08; B01J 6/001; C01B 39/46; B01J 35/1042

(57) ABSTRACT

A molding, comprising a zeolitic material having framework type MFI wherein from 98 to 100 weight-% of the zeolitic material consist of Ti, Si, O, and H, and wherein the zeolitic material having framework type MFI exhibits a type IV nitrogen adsorption/desorption isotherm, the molding further comprising a silica binder, wherein the molding has a pore volume of at least 0.8 mL/g.

The present invention relates to a molding comprising a zeolitic material having framework type MFI wherein from 98 to 100 weight-% of the zeolitic material consist of Ti, Si, O, and H, the process for preparation thereof and its use.

Titanium containing zeolitic materials of structure type MFI are known to be efficient catalysts including, for example, epoxidation reactions. In such industrial-scale processes, typically carried out in continuous mode, these zeolitic materials are usually employed in the form of moldings which, in addition to the catalytically active zeolitic material, comprise a suitable binder.

M. Liu et al. disclose in “Green and efficient preparation of hollow titanium silicalite-1 by using recycled mother liquid” a preparation of hollow titanium silicalite-1 (hollow TS-1, HTS-1) by using recycled mother liquor in the post-synthesis treatment. In this regard, the titanium silicalite-1 starting material was hydrothermally treated with different bases, and hollow cavities were formed in the material. The obtained hollow TS-1 exhibited a better catalytic activity in the propylene epoxidation compared to the starting material.

J. Xu et al. disclose in “Effect of triethylamine treatment of titanium silicalite-1 on propylene epoxidation” in Frontiers of Chemical Science and Engineering a titanium silicalite-1 treated with triethylamine solution under different conditions. It is shown that many irregular hollows are generated in the TS-1 crystals due to the random dissolution of framework silicon. The modified TS-1 samples showed in varying degrees an improved catalyst lifetime when used for the epoxidation of propylene in a fixed-bed reactor.

M. Liu et al. disclose in “Highly Selective Epoxidation of Propylene in a Low-Pressure Continuous Slurry Reactor and the Regeneration of Catalyst” the epoxidation of propylene to propylene oxide with hydrogen peroxide using a micron-sized TS-1 catalyst with hollow structure.

CN 108250161 A relates to a method for oxidizing allyl alcohol wherein a titanium silicalite is used as catalyst. The titanium silicalite is at least partially modified titanium silicalite wherein the modification treatment includes contacting a titanium silicon molecular sieve as a raw material with a liquid containing nitric acid and a peroxide.

CN 103708493 A relates to a titanium silicon molecular sieve having framework structure MFI and the method for preparation thereof.

It was an object of the present invention to provide a novel molding comprising a zeolitic material having framework type MFI, in particular a novel molding comprising a hollow TS-1 zeolite, having advantageous characteristics, in particular an improved propylene oxide selectivity when used as a catalyst or catalyst component, in particular in the epoxidation reaction of propene to pryplene oxide. It was a further object of the present invention to provide a process for the preparation of such a molding, in particular to provide a process resulting in a molding having advantageous properties, preferably when used as a catalyst or catalyst component, specifically in an oxidation or epoxidation reaction. It was a further object of the present invention to provide an improved process for the epoxidation of propene with hydrogen peroxide as oxidizing agent, exhibiting a very low selectivity with respect to by-products and side-products of the epoxidation reaction while, at the same time, allowing for a very high propylene selectivity.

Surprisingly, it was found that such a molding exhibiting said advantageous characteristics can be provided if a given molding comprising a hollow TS-1 zeolite is subjected to a specific post-treatment, resulting in a molding exhibiting, among others, a specific minimum pore volume determined via instrusion mercury porosimetry as described herein. In particular, it has surprisingly been found that a molding can be provided which shows, if used as a catalyst in an epoxidation reaction of propene to propylene oxide and if compared to prior art moldings comprising HTS-1 or TS-1, significantly increased propylene oxide selectivity and yield, and further exhibits excellent life time properties.

Therefore, the present invention relates to a molding, comprising a zeolitic material having framework type MFI wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1, the molding further comprising a silica binder, wherein the molding has a pore volume of at least 0.8 mL/g, determined via Hg porosimetry as described in Reference Example 2.

Further, the present invention relates to process for preparing a molding comprising a zeolitic material having framework type MFI and a silica binder, preferably the molding described above, the process comprising
(i) providing a mixture comprising a silica binder precursor and a zeolitic material having framework type MFI, wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1,
(ii) shaping the mixture obtained from (i), obtaining a precursor of the molding;
(iii) preparing a mixture comprising the precursor of the molding obtained from (ii) and water, and subjecting the mixture to a water treatment under hydrothermal conditions, obtaining a water-treated precursor of the molding;
(iv) calcining the water-treated precursor of the molding in a gas atmosphere, obtaining the molding.

Yet further, the present invention relates to a molding, preferably the molding described above, obtainable or obtained by the process described above.

Yet further, the present invention relates to the use said molding as an adsorbent, an absorbent, a catalyst or a catalyst component, preferably as a catalyst or as a catalyst component.

It is preferred that the zeolitic material having framework type MFI comprises hollow cavities having a diameter of greater than 5.5 Angstrom, preferably in the range of greater than 5.5 Angstrom to smaller than the size of the crystallite of the zeolitic material, determined via TEM as described in Reference Example 3.

It is preferred that from 99 to 100 weight-%, more preferably from 99.5 to 100 weight-%, more preferably from 99.9 to 100 weight-%, of the zeolitic material having framework type MFI consist of Ti, Si, O, and H.

It is preferred that the zeolitic material having framework type MFI has a sodium content, calculated as Na₂O, in the range of from 0 to 0.1 weight-%, more preferably in the range of from 0 to 0.07 weight-%, more preferably in the range of from 0 to 0.05 weight-%, based on the weight of the zeolitic material. It is also preferred that the zeolitic material having framework type MFI has an iron content, calculated as Fe₂O₃, in the range of from 0 to 0.1 weight-%, more preferably in the range of from 0 to 0.07 weight-%, more preferably in the range of from 0 to 0.05 weight-%, based on the weight of the zeolitic material.

Typically, the zeolitic material comprised in the molding of the present invention is in the form of a powder which, as to its particle size distribution, can be prepared, for example, by a specific synthesis process leading to the desired particle size distribution, or a by milling a given zeolitic material, or by spray-drying a suspension comprising a zeolitic material, or by spray-granulation of a suspension comprising a zeolitic material, or by flash drying a suspension comprising a zeolitic material or by microwave drying a suspension comprising a zeolitic material.

The zeolitic material having framework type MFI may preferably have a volume-based particle size distribution characterized by a Dv90 value in the range of from 80 to 200 micrometer, more preferably in the range of from 90 to 175 micrometer, more preferably in the range of from 100 to 150 micrometer, determined as described in Reference Example 5. Further, the zeolitic material having framework type MFI may preferably have a volume-based particle size distribution characterized by a Dv50 value in the range of from 30 to 75 micrometer, more preferably in the range of from 35 to 65 micrometer, more preferably in the range of from 40 to 55 micrometer, determined as described in Reference Example 5. Further, the zeolitic material having framework type MFI may preferably have a volume-based particle size distribution characterized by a Dv10 value in the range of from 1 to 25 micrometer, preferably in the range of from 3 to 20 micrometer, more preferably in the range of from 5 to 15 micrometer, determined as described in Reference Example 5.

No specific restriction exists in view of the Ti content of the zeolitic material having framework type MFI. It is preferred that the zeolitic material having framework type MFI has a Ti content in the range of from 1.3 to 2.1 weight-%, more preferably in the range of from 1.5 to 1.9 weight-%, more preferably in the range of from 1.6 to 1.8 weight-%, calculated as elemental Ti and based on the weight of zeolitic material.

It is preferred that the zeolitic material having framework type MFI exhibits a ²⁹Si solid state NMR spectrum, determined as described in Reference Example 9, having a main resonance in the range of from −108 to −120 ppm, and more preferably having a minor resonance in the range of from −95 to −107 ppm.

Regarding the total pore volume of the molding which is at least 0.8 mL/g, It is preferred that it is in the range of from 0.8 to 1.5 mL/g, more preferably in the range of from 0.9 to 1.4 mL/g, more preferably in the range of from 1.0 to 1.3 mL/g.

Further, no particular restriction applies regarding the shape of the molding. It is preferred that the molding is in the form of a strand, more preferably a strand having a hexagonal, rectangular, quadratic, triangular, oval, or circular cross-section, more preferably a circular cross-section. Preferably, the cross-section has a diameter in the range of from 0.1 to 10 mm, more preferably in the range of from 0.2 to 7 mm, more preferably in the range of from 0.5 to 5 mm, more preferably in the range of from 1 to 3 mm, more preferably in the range of from 1.5 to 2 mm, more preferably in the range of from 1.6 to 1.8 mm.

It is preferred that the molding exhibits a hardness of at least 4 N, more preferably in the range of from 4 to 20 N, more preferably in the range of from 6 to 15 N, more preferably in the range of from 8 to 10 N, determined as described in Reference Example 4.

No particular restriction exist regarding the weight ratio of the zeolitic material having framework type MFI relative to the silica binder in the molding. It is preferred that the weight ratio of the zeolitic material having framework type MFI relative to the silica binder calculated as SiO₂, MFI:SiO₂, is in the range of from 0.5:1 to 10:1, more preferably in the range of from 1:1 to 5:1, more preferably in the range of from 1.5:1 to 4:1, more preferably in the range of from 2:1 to 3:1.

Generally, the molding of the present invention may comprise, in addition to the zeolitic material and the silica binder, one or more further components, such as one or more zeolitic materials other than the zeolitic material having framework type MFI, and/or one or more binder other than the silica binder, for example an alumina binder, a zirconium binder, a ceria binder, a titanic binder, and the like. It is preferred that from 99 to 100 weight-%, more preferably from 99.5 to 100 weight-%, more preferably from 99.9 to 100 weight-% of the molding consist of the zeolitic material having framework type MFI and the silica binder.

It is preferred that the molding has a BET specific surface area in the range of from 300 to 400 m²/g, more preferably in the range of from 325 to 365 m²/g, more preferably in the range of from 340 to 350 m²/g, determined as described in Reference Example 6.

It is preferred that from 50 to 100 weight-%, more preferably from 60 to 100 weight-% of the molding are present in crystalline form.

In particular, the molding disclosed herein exhibits specific properties when used in the catalytic epoxidation of propylene specifically described in Reference Example 10. It is preferred that the molding exhibits a pressure drop rate in the range of from 0.012 to 0.030 bar(abs)/min, more preferably in the range of from 0.015 to 0.025 bar(abs)/min, more preferably in the range of from 0.016 to 0.020 bar(abs)/min, determined as described in Reference Example 10. Further, it is preferred that the molding exhibits a propylene oxide activity of at least 4.5 weight-%, more preferably in the range of from 4.5 to 7 weight-%, more preferably in the range of from 5 to 6 weight-%, determined as described in Reference Example 10.

Further, the molding exhibits specific properties when used in the catalytic epoxidation of propylene specifically described in Reference Example 11. It is preferred that the molding exhibits a propylene oxide selectivity relative to propylene in the range of from 96 to 100%, preferably in the range of from 96.5 to 100%, more preferably in the range of from 97 to 100%, determined in a continuous epoxidation reaction as described in Reference Example 11. In this regard, it is preferred that the molding exhibits said selectivity at a hydrogen peroxide conversion in the range of from 85 to 95%, more preferably in the range of from 87 to 93%, more preferably in the range of from 88 to 92%, wherein more preferably, said selectivity is determined at a time on stream of 200 hours, preferably at a time on stream of 200 and 300 hours, more preferably at a time on stream of 200, 300 and 400 hours, more preferably at a time on stream of 200, 300, 400 and 500 hours, more preferably at a time on stream of 200, 300, 400, 500 and 600 hours, more preferably at a time on stream of 200, 300, 400, 500, 600 and 700 hours, wherein the term “time on stream” refers to the duration of the continuous epoxidation reaction without regeneration of the catalyst.

Furthermore, the present invention relates to a process for preparing a molding comprising a zeolitic material having framework type MFI and a silica binder, preferably a molding as disclosed herein, the process comprising
(i) providing a mixture comprising a silica binder precursor and a zeolitic material having framework type MFI, wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1,
(ii) shaping the mixture obtained from (i), obtaining a precursor of the molding;
(iii) preparing a mixture comprising the precursor of the molding obtained from (ii) and water, and subjecting the mixture to a water treatment under hydrothermal conditions, obtaining a water-treated precursor of the molding;
(iv) calcining the water-treated precursor of the molding in a gas atmosphere, obtaining the molding.

It is preferred that the zeolitic material having framework type MFI comprises hollow cavities having a diameter of greater than 5.5 Angstrom, more preferably in the range of greater than 5.5 Angstrom to smaller than the size of the crystallite of the zeolitic material, determined via TEM as described in Reference Example 3.

Regarding the preparation of the zeolitic material having framework type MFI according to (i), no specific restrictions exist. A preferred process for preparing the zeolitic material having framework type MFI according to (i) may comprise the following steps:
(a) providing a zeolitic material having framework type MFI, wherein from 98 to 100 weight-% of the zeolitic material consist of Ti, Si, O, and H, and wherein the zeolitic material having framework type MFI exhibits a type I nitrogen adsorption/desorption isotherm determined as described in Reference Example 1;
(b) preparing an aqueous mixture comprising the zeolitic material provided in (i) and a zeolite framework type MFI structure directing agent;
(c) subjecting the mixture obtained from (b) to hydrothermal conditions under autogenous pressure, preferably in an autoclave, obtaining a suspension comprising a precursor of the zeolitic material having framework type MFI according to (i), and separating said precursor from the suspension;
(d) optionally calcining the precursor in a gas atmosphere;
(e) subjecting the precursor obtained from (c) or (d) to an acid treatment and preferably drying the acid-treated precursor in a gas atmosphere;
(f) calcining the acid-treated precursor, obtaining the zeolitic material having framework type MFI according to (i).

Preferably, the zeolite framework type MFI structure directing agent according to (a) comprises an tetraalkylammonium salt, preferably tetraalkylammonium hydroxide. In the mixture according to (b), the weight ratio of the zeolite framework type MFI structure directing agent relative to the zeolitic material provided in (i), SDA:MFI, is in the range of from 1:4 to 3:1, more preferably in the range of from 1:2 to 1.5:1, more preferably in the range of from 0.8:1 to 0.9:1. Preferably, the hydrothermal conditions according to (c) comprise a temperature of the mixture in the range of from 150 to 190° C., more preferably in the range of from 160 to 180° C., more preferably in the range of from 165 to 175° C. Subjecting the mixture obtained from (b) to hydrothermal conditions according to (c) preferably may be carried out for 5 to 50 h, preferably for 10 to 30 h, more preferably for 20 to 25 h. Preferably, separating according to (c) comprises subjecting the suspension to filtration or centrifugation, wherein more preferably, separating further comprises washing the zeolitic material having framework type MFI at least once with a liquid solvent system, wherein the liquid solvent system preferably comprises one or more of water, an alcohol, and a mixture of two or more thereof, more preferably water. Preferably, separating according to (c) further comprises drying the precursor, preferably the washed precursor, in a gas atmosphere. Preferably, drying is carried out at a temperature of the gas atmosphere in the range of from 40 to 80° C., more preferably in the range of from 50 to 70° C., more preferably in the range of from 55 to 65° C. Preferably, the gas atmosphere comprises nitrogen, oxygen, or a mixture thereof, wherein the gas atmosphere is more preferably oxygen, air, or lean air. If carried out, calcining of the preursor according according to (d) is preferably carried out at a temperature of the gas atmosphere in the range of from 450 to 550° C., more preferably in the range of from 475 to 525° C., more preferably in the range of from 490 to 510° C. Preferably, the gas atmosphere comprises nitrogen, oxygen, or a mixture thereof, wherein the gas atmosphere is more preferably oxygen, air, or lean air.

Preferably, according to (e), the acid treatment comprises preparing an aqueous mixture of the precursor obtained from (c) or (d) and an acid, and heating the respectively obtained mixture. Regarding the chemical nature of the acid, no specific restrictions exist. Preferably, the acid is one or more nitric acid, sulphuric acid, and acetic acid, more preferably nictric acid. In this aqueous mixture, the weight ratio of the acid relative to the precursor obtained from (c) or (d), is preferably in the range of from 1:2 to 5:1, preferably in the range of from 1:1 to 3:1, more preferably in the range of from 1.5:1 to 2.5:1. Preferably, the aqueous mixture is heated under re-flux, preferably, for example, for 0.5 to 1.5 h, more preferably for 0.75 to 1.25 h. If according to (d), the acid-treated precursor is dried in a gas atmosphere, the drying is preferably carried out at a temperature of the gas atmosphere in the range of from 100 to 140° C., more preferably in the range of from 110 to 130° C. Preferably, the gas atmosphere used for drying comprises nitrogen, oxygen, or a mixture thereof, wherein the gas atmosphere is more preferably oxygen, air, or lean air. Preferably, the drying may be carried out for 2 to 6 h, more preferably 3 to 5 h. According to (f), the calcining is preferably carried out at a temperature of the gas atmosphere in the range of from 450 to 550° C., more preferably in the range of from 475 to 525° C., more preferably in the range of from 490 to 520° C. The calcining may be preferably carried out for 3 to 10 h, more preferably for 5 to 8 h. Preferably, the gas atmosphere used for calcining comprises nitrogen, oxygen, or a mixture thereof, wherein the gas atmosphere is more preferably oxygen, air, or lean air.

The present invention also relates to a zeolitic material having framework type MFI, wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1, obtainable or obtained by a method comprising, preferably consisting of steps (a) to (f).

Therefore, the present invention also relates to a process for preparing a molding as described above, said process comprising preparing a zeolitic material having framework type MFI, wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1, according to a method comprising
(a) providing a zeolitic material having framework type MFI, wherein from 98 to 100 weight-% of the zeolitic material consist of Ti, Si, O, and H, and wherein the zeolitic material having framework type MFI exhibits a type I nitrogen adsorption/desorption isotherm determined as described in Reference Example 1;
(b) preparing an aqueous mixture comprising the zeolitic material provided in (i) and a zeolite framework type MFI structure directing agent;
(c) subjecting the mixture obtained from (b) to hydrothermal conditions under autogenous pressure, preferably in an autoclave, obtaining a suspension comprising a precursor of the zeolitic material having framework type MFI according to (i), and separating said precursor from the suspension;
(d) optionally calcining the precursor in a gas atmosphere;
(e) subjecting the precursor obtained from (c) or (d) to an acid treatment and preferably drying the acid-treated precursor;
(f) calcining the acid-treated precursor, obtaining the zeolitic material having framework type MFI according to (i);
   said process further comprising
(i) preparing a mixture comprising a silica binder precursor and the zeolitic material obtained from (f) or a zeolitic material obtainable or obtained by a method comprising (a) to (c);
(ii) shaping the mixture obtained from (i), obtaining a precursor of the molding;
(iii) preparing a mixture comprising the precursor of the molding obtained from (ii) and water, and subjecting the mixture to a water treatment under hydrothermal conditions, obtaining a water-treated precursor of the molding;
(iv) calcining the water-treated precursor of the molding in a gas atmosphere, obtaining the molding.

As described above, 98 to 100 weight-% of the zeolitic material having framework type MFI comprised in the mixture provided in (i) consist of Ti, Si, O, and H. Thus, the zeolitc material may generally comprise one or more further elements of the periodic system of elements. It is preferred that from 99 to 100 weight-%, more preferably from 99.5 to 100 weight-%, more preferably from 99.9 to 100 weight-%, of the zeolitic material having framework type MFI consist of Ti, Si, O, and H.

It is preferred that the zeolitic material having framework type MFI according to (i) has a sodium content, calculated as Na₂O, in the range of from 0 to 0.1 weight-%, more preferably in the range of from 0 to 0.07 weight-%, more preferably in the range of from 0 to 0.05 weight-%, based on the weight of the zeolitic material. It is preferred that the zeolitic material having framework type MFI has an iron content, calculated as Fe₂O₃, in the range of from 0 to 0.1 weight-%, more preferably in the range of from 0 to 0.07 weight-%, more preferably in the range of from 0 to 0.05 weight-%, based on the weight of the zeolitic material.

Typically, the zeolitic material according to (i) is in the form of a powder which, as to its particle size distribution, can be prepared, for example, by a specific synthesis process leading to the desired particle size distribution, or a by milling a given zeolitic material, or by spray-drying a suspension comprising a zeolitic material, or by spray-granulation of a suspension comprising a zeolitic material, or by flash drying a suspension comprising a zeolitic material or by microwave drying a suspension comprising a zeolitic material.

The zeolitic material having framework type MFI according to (i) may preferably have a volume-based particle size distribution characterized by a Dv90 value in the range of from 80 to 200 micrometer, more preferably in the range of from 90 to 175 micrometer, more preferably in the range of from 100 to 150 micrometer, determined as described in Reference Example 5. Further, the zeolitic material having framework type MFI may preferably have a volume-based particle size distribution characterized by a Dv50 value in the range of from 30 to 75 micrometer, more preferably in the range of from 35 to 65 micrometer, more preferably in the range of from 40 to 55 micrometer, determined as described in Reference Example 5. Further, the zeolitic material having framework type MFI may preferably have a volume-based particle size distribution characterized by a Dv10 value in the range of from 1 to 25 micrometer, preferably in the range of from 3 to 20 micrometer, more preferably in the range of from 5 to 15 micrometer, determined as described in Reference Example 5.

No specific restriction exists in view of the Ti content of the zeolitic material having framework type MFI according to (i). It is preferred that the zeolitic material having framework type MFI has a Ti content in the range of from 1.3 to 2.1 weight-%, more preferably in the range of from 1.5 to 1.9 weight-%, more preferably in the range of from 1.6 to 1.8 weight-%, calculated as elemental Ti and based on the weight of zeolitic material.

It is preferred that the zeolitic material having framework type MFI according to (i) exhibits a ²⁹Si solid state NMR spectrum, determined as described in Reference Example 9, having a main resonance in the range of from −108 to −120 ppm, and more preferably having a minor resonance in the range of from −95 to −107 ppm.

Preferably, the zeolitic material according to (i) has a BET specific surface area of at least 300 m²/g, more preferably in the range of from 350 to 500, preferably in the range of from 375 to 450, more preferably in the range of from 390 to 410, determined as described in Reference Example 6.

It is preferred that the silica binder precursor is selected from the group consisting of a silica sol, a colloidal silica, a wet process silica, a dry process silica, and a mixture of two or more thereof, wherein the silica binder precursor is more preferably a colloidal silica. In this context, both colloidal silica and so-called “wet process” silica and so-called “dry process” silica can be used. Colloidal silica, preferably as an alkaline and/or ammoniacal solution, more preferably as an ammoniacal solution, is commercially available, inter alia, for example as Ludox®, Syton®, Nalco® or Snowtex®. “Wet process” silica is commercially available, inter alia, for example as Hi-Sil®, Ultrasil®, Vulcasil®, Santocel®, Valron-Estersil®, Tokusil® or Nipsil®. “Dry process” silica is commercially available, inter alia, for example as Aerosil®, Reolosil®, Cab-O-Sil®, Fransil® or ArcSilica®. An ammoniacal solution of colloidal silica is preferred according to the present invention.

As regards the physical or chemical nature of the mixture provided in (i), no particular restriction applies. It is preferred that in the mixture according to (i), the weight ratio of zeolitic material, relative to Si comprised in the silica binder precursor, calculated as SiO₂, is in the range of from 0.5:1 to 10:1, more preferably in the range of from 1:1 to 5:1, more preferably in the range of from 1.5:1 to 4:1, more preferably in the range of from 2:1 to 3:1.

Preferably, the mixture provided in (i) comprises one or more further components in addition to the zeolitic material and the silica binder precursor. More preferably, the mixture according to (i) further comprises one or more viscosity modifying agents, or one or more pore forming agents, preferably more mesopore forming agents, or one or more viscosity modifying agents and one or more pore forming agents, preferably mesopore forming agents.

It is preferred that the one or more agents are selected from the group consisting of water, alcohols, organic polymers, and mixtures of two or more thereof, wherein the organic polymers are more preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose derivatives, polyalkylene oxides, polystyrenes, and mixtures of two or more thereof, wherein the organic polymers are more preferably selected from the group consisting of a metyhl celluloses, carboxymethylcelluloses, polyethylene oxides, polystyrenes, and mixtures of two or more thereof, wherein more preferably, the one or more agents comprise water, a carboxymethylcellulose, a polyethylene oxide, and a polystyrene. More preferably, the one or more agents consist of water, a carboxymethylcellulose, a polyethylene oxide, and a polystyrene.

If the mixture prepared according to (i) comprises one or more viscosity modifying and/or mesopore forming agents, the weight ratio of zeolitic material, relative to the one or more agents, i.e. to all of these agents, is preferably in the range of from 1:1 to 5:1, preferably in the range of from 2.5:1 to 4:1, more preferably in the range of from 3:1 to 3.5:1.

As regards the provision of the mixture in (i), i.e. the method how the mixture it prepared, no particular restrictions exist. It is preferred that the mixture is prepared by suitably mixing the respective components, preferably by mixing in a kneader or in a mix-muller.

Depending on the mass of the mixture, the mixing or kneading time should be adjusted. For illustrative purpose only, the mixture may for example be mixed or kneaded for 15 to 60 min, preferably for 30 to 55 min, more preferably for 40 to 50 min.

As regards the shaping of the mixture obtained from (i) according to (ii), no particular restriction applies as long as a precursor of the molding can be obtained. Thus, the mixture obtained from (i) can be shaped to any conceivable form. It is preferred that in (ii), the mixture is shaped to a strand, more preferably to a strand having a circular cross-section.

In the case where the the mixture is shaped according to (ii) to a strand having a circular cross-section, no particular restriction applies in view of the diameter of the circular cross-section. It is preferred that the strand having a circular cross-section has a diameter in the range of from 0.1 to 10 mm, more preferably in the range of from 0.2 to 7 mm, more preferably in the range of from 0.5 to 5 mm, more preferably in the range of from 1 to 3 mm, more preferably in the range of from 1.5 to 2 mm, more preferably in the range of from 1.6 to 1.8 mm.

As regards shaping in (ii), no particular restriction applies such that shaping may be performed by any conceivable means. It is preferred that in (ii), shaping comprises extruding the mixture.

Suitable extrusion apparatuses are described, for example, in “Ullmann's Enzyklopädie der Technischen Chemie”, 4th edition, vol. 2, page 295 et seq., 1972. In addition to the use of an extruder, an extrusion press can also be used for the preparation of the moldings. If necessary, the extruder can be suitably cooled during the extrusion process. The strands leaving the extruder via the extruder die head can be mechanically cut by a suitable wire or via a discontinuous gas stream.

As regards shaping in (ii), no particular restriction applies such that (ii) may comprise further steps. It is preferred that (ii) further comprises drying of the precursor of the molding in a gas atmosphere.

In the case where the precursor of the molding is dried in a gas atmosphere, no particular restriction applies in view of the conditions of drying. It is preferred that drying is carried out at a temperature of the gas atmosphere in the range of from 80 to 160° C., more preferably in the range of from 100 to 140° C., more preferably in the range of from 110 to 130° C.

Depending on the mass of the precursor of the molding to be dried, the duration of drying should be adjusted. For illustrative purpose only, the precursor of the molding may for example be dried in a gas atmosphere for 2 to 6 h, preferably for 3 to 5 h, more preferably for 3.5 to 4.5 h.

Further in the case where the precursor of the molding is dried in a gas atmosphere, no particular restriction applies in view of the gas atmosphere for drying. It is preferred that the gas atmosphere comprises nitrogen, oxygen, or a mixture thereof, wherein the gas atmosphere is more preferably oxygen, air, or lean air.

As disclosed above, no particular restriction applies as regards shaping in (ii), such that (ii) may comprise further steps. It is preferred that (ii) further comprises calcining of the precursor of the molding in a gas atmosphere, preferably of the dried precursor of the molding.

In the case where the precursor of the molding is calcined in a gas atmosphere, no particular restriction applies in view of the conditions of calcining. It is preferred that calcining is carried out at a temperature of the gas atmosphere in the range of from 450 to 530° C., more preferably in the range of from 470 to 510° C., more preferably in the range of from 480 to 500° C.

Depending on the mass of the precursor of the molding to be calcined, the duration of calcining should be adjusted. For illustrative purpose only, the precursor of the molding may for example be calcined in a gas atmosphere for 3 to 7 h, preferably for 4 to 6 h, more preferably for 4.5 to 5.5 h.

Further in the case where the precursor of the molding is calcined in a gas atmosphere, no particular restriction applies in view of the gas atmosphere for calcining. It is preferred that the gas atmosphere comprises nitrogen, oxygen, or a mixture thereof, wherein the gas atmosphere is preferably oxygen, air, or lean air.

As regards the conditions of the water treatment according to (iii), no particular restriction applies such that any conveivable conditions may be applied as long as the water treatment includes hydrothermal conditions and as long as a water-treated precursor of the molding can be obtained. It is preferred that the water treatment according to (iii) comprises a temperature of the mixture in the range of from 100 to 200° C., more preferably in the range of from 125 to 175° C., more preferably in the range of from 130 to 160° C., more preferably in the range of from 135 to 155° C. more preferably in the range of from 140 to 150° C.

As disclosed above, no particular restriction applies in view of the conditions of the water treatment according to (iii). It is preferred that the water treatment according to (iii) is carried out under autogenous pressure. It is particularly preferred that the water treatment according to (iii) is carried out in an autoclave.

As disclosed above, no particular restriction applies in view of the conditions of the water treatment according to (iii). It is preferred that the water treatment according to (iii) is carried out for 6 to 10 h, more preferably for 7 to 9 h, more preferably for 7.5 to 8.5 h.

As regards the physical or chemical nature of the mixture prepared in (iii), no particular restriction applies. It is preferred that in the mixture prepared in (iii), the weight ratio of the zeolitic material relative to the water is in the range of from 1:1 to 1:10, more preferably in the range of from 1:3 to 1:7, more preferably in the range of from 1:4 to 1:6.

As regards the process comprising (i), (ii), (iii) and (iv) as disclosed herein, no particular restriction applies in view of further process steps that may in particular be carried out between two steps. It is preferred that after (iii) and prior to (iv), the water-treated precursor of the molding is separated from the mixture obtained from (iii), wherein separating preferably comprises subjecting the mixture obtained from (iii) to filtration or centrifugation, wherein more preferably, separating further comprises washing the water-treated precursor of the molding at least once with a liquid solvent system, wherein the liquid solvent system preferably comprises one or more of water, an alcohol, and a mixture of two or more thereof, wherein the water-treated precursor of the molding is more preferably washed with water.

As disclosed above, the process may comprise further steps. It is preferred that after (iii) and prior to (iv), the water-treated precursor of the molding is dried in a gas atmosphere, wherein the water-treated precursor of the molding is preferably separated according to the above. With regard to the drying, no particular restriction applies in view of the conditions of drying. It is preferred that drying is carried out at a temperature of the gas atmosphere in the range of from 80 to 160° C., more preferably in the range of from 100 to 140° C., more preferably in the range of from 110 to 130° C.

Depending on the mass of the water-treated precursor of the molding to be dried, the duration of drying should be adjusted. For illustrative purpose only, the precursor of the molding may for example be dried in a gas atmosphere for 2 to 6 h, preferably for 3 to 5 h, more preferably for 3.5 to 4.5 h.

Further in the case where the water-treated precursor of the molding or the separated water-treated precursor of the molding is dried in a gas atmosphere, no particular restriction applies in view of the gas atmosphere for drying. It is preferred that the gas atmosphere comprises nitrogen, oxygen, or a mixture thereof, wherein the gas atmosphere is preferably oxygen, air, or lean air.

As regards the conditions of calcining in (iv), no particular restriction applies. It is preferred that calcining in (iv) is carried out at a temperature of the gas atmosphere in the range of from 400 to 490° C., more preferably in the range of from 420 to 470° C., more preferably in the range of from 440 to 460° C.

Depending on the mass of the water-treated precursor of the molding to be calcined, the duration of calcining should be adjusted. For illustrative purpose only, the precursor of the molding may for example be calcined in a gas atmosphere for 0.5 to 5 h, preferably for 1 to 3 h, more preferably for 1.5 to 2.5 h.

Further, no particular restriction applies in view of the gas atmosphere for calcining. It is preferred that the gas atmosphere according to (iv) comprises nitrogen, oxygen, or a mixture thereof, wherein the gas atmosphere is preferably oxygen, air or lean air.

It is particularly preferred to carry out the calcination in a muffle furnace, a rotary kiln and/or a belt calcining furnace.

Furthermore, the present invention relates to a molding comprising a zeolitic material having framework type MFI and a silica binder, obtainable or obtained by a process as described hereinabove.

Furthermore, the present invention relates to a use of a molding according to any one of the embodiments disclosed herein as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component, more preferably as an oxidation catalyst or as an oxidation catalyst component, more preferably as an epoxidation catalyst or as an epoxidation catalyst component, more preferably as an epoxidation catalyst.

The present invention also relates to the use of said molding as a catalyst or catalyst component, preferably as a catalyst component for preparing propylene oxide from propene with hydrogen peroxide as oxidizing agent in methanol as solvent.

According to the present invention, it is conceivable that if hydrogen peroxide is used as oxidizing agent, the hydrogen peroxide is formed in situ during the reaction from hydrogen and oxygen or from other suitable precursors. More preferably, the term “using hydrogen peroxide as oxidizing agent” as used in the context of the present invention relates to an embodiment where hydrogen peroxide is not formed in situ but employed as starting material, preferably in the form of a solution, preferably an at least partially aqueous solution, more preferably an aqueous solution, said preferably aqueous solution having a preferred hydrogen peroxide concentration in the range of from 20 to 60, more preferably from 25 to 55 weight-%, based on the total weight of the solution.

In addition to the above, the present invention relates to a process for the preparation of propylene oxide wherein the inventive molding is used as a catalytic species. According to the process, propene is reacted with hydrogen peroxide in methanolic solution in the presence of a molding as disclosed herein to obtain propylene oxide. The reaction feed which is introduced in the at least one reactor in which the inventive continuous epoxidation process is carried out comprises propene, methanol and hydrogen peroxide. Further, this reaction feed comprises a specific amount of potassium cations and additionally phosphorus in the form of anions of at least one phosphorus oxyacid.

Conversion and selectivity of epoxidation reactions can be influenced, for example, via the temperature of the epoxidation reaction, the pH of the epoxidation reaction mixture, and/or addition of one or more compounds to the reaction mixture other than the reactants propene and hydrogen peroxide.

If the molding is used as an epoxidation catalyst or as an epoxidation catalyst component for an epoxidation reaction of an organic compound, no particular restriction applies in view of the organic compound. It is preferred that the organic compound has at least one C—C double bond, wherein the organic compound is more preferably a C2-C10 alkene, more preferably a C2-C5 alkene, more preferably a C2-C4 alkene, more preferably a C2 or C3 alkene, more preferably propene. It is particularly preferred that the molding is used as an epoxidation catalyst or as an epoxidation catalyst component for the epoxidation of propene, more preferably for the epoxidation of propene with hydrogen peroxide as oxidizing agent, more preferably for the epoxidation of propene with hydrogen peroxide as oxidizing agent in a solvent comprising an alcohol, preferably methanol.

According to the present invention, it is conceivable that if hydrogen peroxide is used as oxidizing agent, the hydrogen peroxide is formed in situ during the reaction from hydrogen and oxygen or from other suitable precursors. However, most preferably, the term “using hydrogen peroxide as oxidizing agent” as used in the context of the present invention relates to an embodiment where hydrogen peroxide is not formed in situ but employed as starting material, preferably in the form of a solution, preferably an at least partially aqueous solution, more preferably an aqueous solution, said preferably aqueous solution having a preferred hydrogen peroxide concentration in the range of from 20 to 60, more preferably from 25 to 55 weight-%, based on the total weight of the solution.

Furthermore, the present invention relates to a process for oxidizing an organic compound comprising bringing the organic compound in contact with a catalyst comprising a molding according to any one of the embodiments disclosed herein, preferably for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene.

As regards the conditions of the process for oxidizing an organic compound, no particular restriction applies such that one or more agents may be used therein. It is preferred that the process comprises use of an oxidizing agent, wherein more preferably hydrogen peroxide is used as oxidizing agent, and wherein the oxidation reaction is more preferably carried out in a solvent, more preferably in a solvent comprising an alcohol, preferably methanol.

Furthermore, the present invention relates to a process for preparing propylene oxide comprising reacting propene with hydrogen peroxide in methanolic solution in the presence of a catalyst comprising a molding according to any one of the embodiments disclosed herein to obtain propylene oxide.

Typically, the process for preparing propylene oxide is carried out in a reactor. As regards the means for providing propene, methanol and hydrogen peroxide into the reactor, no particular restriction applies. It is preferred that a reaction feed comprising propene, methanol and hydrogen peroxide is introduced into a reactor, said reaction feed containing potassium cations (K⁺) in an amount of from 100 to 160 micromol, relative to 1 mol hydrogen peroxide contained in the reaction feed, and further containing phosphorus (P) in the form of anions of at least one phosphorus oxyacid.

As regards the physical or chemical nature of the reaction feed, no particular restriction applies such that it may comprise one or more further components. It is preferred that the reaction feed contains K⁺ in an amount of from 100 to 155 micromol, more preferably of from 120 to 150 micromol, relative to 1 mol hydrogen peroxide contained the reaction feed.

As disclosed above, no particular restriction applies in view of the physical or chemical nature of the reaction feed. It is preferred that in the reaction feed, the molar ratio of K⁺ relative to P is in the range of from 1.5:1 to 2.5:1, more preferably of from 1.9:1 to 2.1:1.

As disclosed above, no particular restriction applies in view of the physical or chemical nature of the reaction feed. It is preferred that the reaction feed is obtained from a hydrogen peroxide feed, a methanol feed, and a propene feed.

In the case where the reaction feed is obtained from a hydrogen peroxide feed, a methanol feed, and a propene feed, no particular restriction applies in view of the physical or chemical nature of the hydrogen peroxide feed. It is preferred that the hydrogen peroxide feed contains K⁺ in an amount of less than 110 micromol, more preferably less than 70 micromol, more preferably less than 30 micromol, in particular less than 5 micromol, relative to 1 mol hydrogen peroxide contained in the hydrogen peroxide feed.

In the case where the hydrogen peroxide feed contains K⁺ in an amount of less than 110 micromol, again no particular restriction applies in view of the physical or chemical nature of the hydrogen peroxide feed. It is preferred that at least one solution containing K⁺ and P in the form of anions of at least one phosphorus oxyacid is added to the hydrogen peroxide feed or to the propene feed or to the methanol feed or a mixed feed of two or three thereof, in such an amount that the reaction feed contains K⁺, and P in the form of anions of at least one phosphorus oxyacid in amounts as defined in any one of the respective embodiments disclosed herein.

In the case where at least one solution containing K⁺ and P in the form of anions of at least one phosphorus oxyacid is added to the hydrogen peroxide feed or to the propene feed or to the methanol feed or a mixed feed of two or three thereof, in such an amount that the reaction feed contains K⁺, and P in the form of anions of at least one phosphorus oxyacid in amounts as defined in any one of the respective embodiments disclosed herein, no particular restriction applies in view of the physical or chemical nature of the at least one solution. It is preferred that the at least one solution is an aqueous solution of dipotassium hydrogen phosphate.

In the case where the reaction feed is obtained from a hydrogen peroxide feed, a methanol feed, and a propene feed, no particular restriction applies in view of the physical or chemical nature of the hydrogen peroxide feed. It is preferred that the hydrogen peroxide feed is an aqueous or a methanolic or an aqueous/methanolic, more preferably an aqueous hydrogen peroxide feed, containing hydrogen peroxide preferably in an amount of from 25 to 75 wt.-%, more preferably of from 30 to 50 wt.-%.

In the case where the reaction feed is obtained from a hydrogen peroxide feed, a methanol feed, and a propene feed, no particular restriction applies in view of the physical or chemical nature of the propene feed. It is preferred that the propene feed additionally contains propane wherein the volume ratio of propene to propane is preferably in the range of from 99.99:0.01 to 95:5.

As regards the physical or chemical nature of the reaction feed, no particular restriction applies. Thus, the reaction feed may consist of one or more phases. It is preferred that the reaction feed when introduced into the reactor consists of one liquid phase.

As regards the conditions under which the process for preparing propylene oxide comprising reacting propene with hydrogen peroxide in methanolic solution in the presence of a catalyst comprising a molding is carried out, no particular restriction applies. It is preferred that the pressure under which the reaction of propene with hydrogen peroxide in methanolic solution in the presence of the catalyst comprising the molding is carried out in the reactor is at least 10 bar(abs), more preferably at least 15 bar(abs), more preferably at least 20 bar(abs), more preferably in the range of from 20 to 40 bar(abs).

As disclosed above, no particular restriction applies in view of the conditions under which the process for preparing propylene oxide comprising reacting propene with hydrogen peroxide in methanolic solution in the presence of a catalyst comprising a molding is carried out. It is preferred that the reaction mixture in the reactor is externally and/or internally cooled such that the maximum temperature of the reaction mixture in the reactor is in the range of from 30 to 70° C.

As regards the method for carrying out the reaction of propene with hydrogen peroxide in methanolic solution in the presence of the catalyst comprising the molding, no particular restriction applies. It is preferred that the reaction of propene with hydrogen peroxide in methanolic solution in the presence of the catalyst comprising the molding is carried out by a method comprising
(a) reacting propene with hydrogen peroxide in methanolic solution in the presence of a catalyst comprising the molding in at least one reactor R1 which is preferably operated in isothermal mode, wherein a reaction feed comprising propene, methanol and hydrogen peroxide is introduced into R1, said reaction feed containing potassium cations (K⁺) in an amount of from 100 to 160 micromol, relative to 1 mol hydrogen peroxide contained in the reaction feed, and further containing phosphorus (P) in the form of anions of at least one phosphorus oxyacid;
(b) separating a stream containing non-reacted hydrogen peroxide from the reaction mixture obtained from (a) and removed from R1, said separating preferably being carried out by distillation in at least 1, preferably 1 distillation column K1;
(c) mixing the stream containing non-reacted hydrogen peroxide with a propene stream, passing the mixed stream into at least 1, preferably 1 reactor R2 containing a catalyst comprising the molding and preferably being operated in adiabatic mode, and reacting propene with hydrogen peroxide in R2;
   wherein the hydrogen peroxide conversion in R1 is preferably in the range of from 85 to 95%, more preferably in the range of from 87 to 93%.

The unit bar(abs) refers to an absolute pressure of 10⁵ Pa and the unit Angstrom refers to a length of 10⁻¹⁰ m.

The present invention is further illustrated by the following set of embodiments and combinations of embodiments resulting from the dependencies and back-references as indicated. In particular, it is noted that in each instance where a range of embodiments is mentioned, for example in the context of a term such as “The molding of any one of embodiments 1 to 4”, every embodiment in this range is meant to be explicitly disclosed for the skilled person, i.e. the wording of this term is to be understood by the skilled person as being synonymous to “The molding of any one of embodiments 1, 2, 3, and 4”.
1. A molding, comprising a zeolitic material having framework type MFI wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1, the molding further comprising a silica binder, wherein the molding has a pore volume of at least 0.8 mL/g, determined via Hg porosimetry as described in Reference Example 2.
2. The molding of embodiment 1, wherein the zeolitic material having framework type MFI comprises hollow cavities having a diameter of greater than 5.5 Angstrom, preferably in the range of greater than 5.5 Angstrom to smaller than the size of the crystallite of the zeolitic material, determined via TEM as described in Reference Example 3.
3. The molding of embodiment 1 or 2, wherein from 99 to 100 weight-%, preferably from 99.5 to 100 weight-%, more preferably from 99.9 to 100 weight-%, of the zeolitic material having framework type MFI consist of Ti, Si, O, and H.
4. The molding of any one of embodiments 1 to 3, wherein the zeolitic material having framework type MFI has a sodium content, calculated as Na₂O, in the range of from 0 to 0.1 weight-%, preferably in the range of from 0 to 0.07 weight-%, more preferably in the range of from 0 to 0.05 weight-%, based on the weight of the zeolitic material.
5. The molding of any one of embodiments 1 to 4, wherein the zeolitic material having framework type MFI has an iron content, calculated as Fe₂O₃, in the range of from 0 to 0.1 weight-%, preferably in the range of from 0 to 0.07 weight-%, more preferably in the range of from 0 to 0.05 weight-%, based on the weight of the zeolitic material.
6. The molding of any one of embodiments 1 to 5, wherein the zeolitic material having framework type MFI has a volume-based particle size distribution characterized by a Dv90 value in the range of from 80 to 200 micrometer, preferably in the range of from 90 to 175 micrometer, more preferably in the range of from 100 to 150 micrometer, determined as described in Reference Example 5.
7. The molding of any one of embodiments 1 to 6, wherein the zeolitic material having framework type MFI has a volume-based particle size distribution characterized by a Dv50 value in the range of from 30 to 75 micrometer, preferably in the range of from 35 to 65 micrometer, more preferably in the range of from 40 to 55 micrometer, determined as described in Reference Example 5.
8. The molding of any one of embodiments 1 to 7, wherein the zeolitic material having framework type MFI has a volume-based particle size distribution characterized by a Dv10 value in the range of from 1 to 25 micrometer, preferably in the range of from 3 to 20 micrometer, more preferably in the range of from 5 to 15 micrometer, determined as described in Reference Example 5.

9. The molding of any one of embodiments 1 to 8, wherein the zeolitic material having framework type MFI has a Ti content in the range of from 1.3 to 2.1 weight-%, preferably in the range of from 1.5 to 1.9 weight-%, more preferably in the range of from 1.6 to 1.8 weight-%, calculated as elemental Ti and based on the weight of zeolitic material.
10. The molding of any one of embodiments 1 to 9, wherein the zeolitic material having framework type MFI exhibits a ²⁹Si solid state NMR spectrum, determined as described in Reference Example 9, having a main resonance in the range of from −108 to −120 ppm, and preferably having a minor resonance in the range of from −95 to −107 ppm.
11. The molding of any one of embodiments 1 to 10, wherein the pore volume is in the range of from 0.8 to 1.5 mL/g, preferably in the range of from 0.9 to 1.4 mL/g, more preferably in the range of from 1.0 to 1.3 mL/g.
12. The molding of any one of embodiments 1 to 11, being a strand, preferably having a hexagonal, rectangular, quadratic, triangular, oval, or circular cross-section, more preferably a circular cross-section, wherein the cross-section has a diameter preferably in the range of from 0.5 to 5 mm, more preferably in the range of from 1 to 3 mm, more preferably in the range of from 1.5 to 2 mm, more preferably in the range of from 1.6 to 1.8 mm.
13. The molding of any one of embodiments 1 to 12, exhibiting a hardness of at least 4 N, preferably in the range of from 4 to 20 N, more preferably in the range of from 6 to 15 N, more preferably in the range of from 8 to 10 N, determined as described in Reference Example 4.
14. The molding of any one of embodiments 1 to 13, wherein in the molding, the weight ratio of the zeolitic material having framework type MFI relative to the silica binder calculated as SiO₂, MFI:SiO₂, is in the range of from 1:1 to 5:1, preferably in the range of from 1.5:1 to 4:1, more preferably in the range of from 2:1 to 3:1.
15. The molding of any one of embodiments 1 to 14, wherein from 99 to 100 weight-%, preferably from 99.5 to 100 weight-%, more preferably from 99.9 to 100 weight-% of the molding consist of the zeolitic material having framework type MFI and the silica binder.
16. The molding of any one of embodiments 1 to 15, having a BET specific surface area in the range of from 300 to 400, preferably in the range of from 325 to 365, more preferably in the range of from 340 to 350, determined as described in Reference Example 6.
17. The molding of any one of embodiments 1 to 16, wherein from 50 to 100 weight-%, preferably from 60 to 100 weight-% of the molding are present in crystalline form.
18. The molding of any one of embodiments 1 to 17, exhibiting a pressure drop rate in the range of from 0.012 to 0.030 bar(abs)/min, preferably in the range of from 0.015 to 0.025 bar(abs)/min, more preferably in the range of from 0.016 to 0.020 bar(abs)/min, determined as described in Reference Example 10.
19. The molding of any one of embodiments 1 to 18, exhibiting a propylene oxide activity of at least 4.5 weight-%, preferably in the range of from 4.5 to 7 weight-%, more preferably in the range of from 5 to 6 weight-%, determined as described in Reference Example 10.
20. The molding of any one of embodiments 1 to 19, exhibiting a propylene oxide selectivity relative to propylene in the range of from 96 to 100%, preferably in the range of from 96.5 to 100%, more preferably in the range of from 97 to 100%, determined in a continuous epoxidation reaction as described in Reference Example 11.
21. The molding of embodiment 20, exhibiting said selectivity at a hydrogen peroxide conversion in the range of from 85 to 95%, preferably in the range of from 87 to 93%, more preferably in the range of from 88 to 92%, wherein more preferably, said selectivity is determined at a time on stream of 200 hours, preferably at a time on stream of 200 and 300 hours, more preferably at a time on stream of 200, 300 and 400 hours, more preferably at a time on stream of 200, 300, 400 and 500 hours, more preferably at a time on stream of 200, 300, 400, 500 and 600 hours, more preferably at a time on stream of 200, 300, 400, 500, 600 and 700 hours, wherein the term “time on stream” refers to the duration of the continuous epoxidation reaction without regeneration of the catalyst.
22. A process for preparing a molding comprising a zeolitic material having framework type MFI and a silica binder, preferably a molding according to any one of embodiments 1 to 21, the process comprising
   (i) providing a mixture comprising a silica binder precursor and a zeolitic material having framework type MFI, wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1;
   (ii) shaping the mixture obtained from (i), obtaining a precursor of the molding;
   (iii) preparing a mixture comprising the precursor of the molding obtained from (ii) and water, and subjecting the mixture to a water treatment under hydrothermal conditions, obtaining a water-treated precursor of the molding;
   (iv) calcining the water-treated precursor of the molding in a gas atmosphere, obtaining the molding.
23. The process of embodiment 22, wherein the zeolitic material having framework type MFI comprises hollow cavities having a diameter of greater than 5.5 Angstrom, preferably in the range of greater than 5.5 Angstrom to smaller than the size of the crystallite of the zeolitic material, determined via TEM as described in Reference Example 3.
24. The process of embodiment 22 or 23, wherein from 99 to 100 weight-%, preferably from 99.5 to 100 weight-%, more preferably from 99.9 to 100 weight-%, of the zeolitic material having framework type MFI consist of Ti, Si, O, and H.
25. The process of any one of embodiments 22 to 24, wherein the zeolitic material having framework type MFI has a sodium content, calculated as Na₂O, in the range of from 0 to 0.1 weight-%, preferably in the range of from 0 to 0.07 weight-%, more preferably in the range of from 0 to 0.05 weight-%, based on the weight of the zeolitic material.
26. The process of any one of embodiments 22 to 25, wherein the zeolitic material having framework type MFI has an iron content, calculated as Fe₂O₃, in the range of from 0 to 0.1 weight-%, preferably in the range of from 0 to 0.07 weight-%, more preferably in the range of from 0 to 0.05 weight-%, based on the weight of the zeolitic material.
27. The process of any one of embodiments 22 to 26, wherein the zeolitic material having framework type MFI has a volume-based particle size distribution characterized by a Dv90 value in the range of from 80 to 200 micrometer, preferably in the range of from 90 to 175 micrometer, more preferably in the range of from 100 to 150 micrometer, determined as described in Reference Example 5.
28. The process of any one of embodiments 22 to 27, wherein the zeolitic material having framework type MFI has a volume-based particle size distribution characterized by a Dv50 value in the range of from 30 to 75 micrometer, preferably in the range of from 35 to 65 micrometer, more preferably in the range of from 40 to 55 micrometer, determined as described in Reference Example 5.
29. The process of any one of embodiments 22 to 28, wherein the zeolitic material having framework type MFI has a volume-based particle size distribution characterized by a Dv10 value in the range of from 1 to 25 micrometer, preferably in the range of from 3 to 20 micrometer, more preferably in the range of from 5 to 15 micrometer, determined as described in Reference Example 5.
30. The process of any one of embodiments 22 to 29, wherein the zeolitic material having framework type MFI has a Ti content in the range of from 1.3 to 2.1 weight-%, preferably in the range of from 1.5 to 1.9 weight-%, more preferably in the range of from 1.6 to 1.8 weight-%, calculated as elemental Ti and based on the weight of zeolitic material.
31. The process of any one of embodiments 22 to 30, wherein the zeolitic material having framework type MFI exhibits a ²⁹Si solid state NMR spectrum, determined as described in Reference Example 9, having a main resonance in the range of from −108 to −120 ppm, and preferably having a minor resonance in the range of from −95 to −107 ppm.
32. The process of any one of embodiments 22 to 31, wherein the zeolitic material having framework type MFI has a BET specific surface area of at least 300 m²/g, preferably in the range of from 350 to 500 m²/g, preferably in the range of from 375 to 450 m²/g, more preferably in the range of from 390 to 410 m²/g, determined as described in Reference Example 6.
33. The process of any one of embodiments 22 to 32, wherein the silica binder precursor is selected from the group consisting of a silica sol, a colloidal silica, a wet process silica, a dry process silica, and a mixture of two or more thereof, wherein the silica binder precursor is preferably a colloidal silica.
34. The process of any one of embodiments 22 to 33, wherein in the mixture according to (i), the weight ratio of zeolitic material, relative to Si comprised in the silica binder precursor, calculated as SiO₂, is in the range of from 1:1 to 5:1, preferably in the range of from 1.5:1 to 4:1, more preferably in the range of from 2:1 to 3:1.
35. The process of any one of embodiments 22 to 34, wherein the mixture prepared according to (i) further comprises one or more viscosity modifying and/or mesopore forming agents.
36. The process of embodiment 35, wherein the one or more agents are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose derivatives, polyalkylene oxides, polystyrenes, and mixtures of two or more thereof, wherein the organic polymers are more preferably selected from the group consisting of a metyhl celluloses, carboxymethylcelluloses, polyethylene oxides, polystyrenes, and mixtures of two or more thereof, wherein more preferably, the one or more agents comprise water, a carboxymethylcellulose, a polyethylene oxide, and a polystyrene.
37. The process of embodiment 35 or 36, wherein in the mixture prepared according to (i), the weight ratio of zeolitic material, relative to the one or more agents is in the range of from 1:1 to 5:1, preferably in the range of from 2.5:1 to 4:1, more preferably in the range of from 3:1 to 3.5:1.
38. The process of any one of embodiments 22 to 37, wherein the mixture is mixed in a kneader or in a mix-muller.
39. The process of any one of embodiments 22 to 38, wherein in (ii), the mixture is shaped to a strand, preferably to a strand having a circular cross-section.
40. The process of embodiment 39, wherein the strand having a circular cross-section has a diameter in the range of from 0.2 to 10 mm, preferably in the range of from 0.5 to 5 mm, more preferably in the range of from 1 to 3 mm, more preferably in the range of from 1.5 to 2 mm, more preferably in the range of from 1.6 to 1.8 mm.
41. The process of any one of embodiments 22 to 40, wherein in (ii), shaping comprises extruding the mixture.
42. The process of any one of embodiments 22 to 41, wherein shaping according to (ii) further comprises drying the precursor of the molding in a gas atmosphere.
43. The process of embodiment 42, wherein drying is carried out at a temperature of the gas atmosphere in the range of from 80 to 160° C., preferably in the range of from 100 to 140° C., more preferably in the range of from 110 to 130° C.
44. The process of embodiments 42 or 43, wherein the gas atmosphere comprises nitrogen, oxygen, or a mixture thereof, wherein the gas atmosphere is preferably oxygen, air, or lean air.
45. The process of any one of embodiments 22 to 44, preferably any one of embodiment 37 to 39, wherein shaping according to (ii) further comprises calcining the preferably dried precursor of the molding in a gas atmosphere.
46. The process of embodiment 45, wherein calcining is carried out at a temperature of the gas atmosphere in the range of from 450 to 530° C., preferably in the range of from 470 to 510° C., more preferably in the range of from 480 to 500° C.
47. The process of embodiment 45 or 46, wherein the gas atmosphere comprises nitrogen, oxygen, or a mixture thereof, wherein the gas atmosphere is preferably oxygen, air, or lean air.
48. The process of any one of embodiments 22 to 47, wherein the water treatment according to (iii) comprises a temperature of the mixture in the range of from 100 to 200° C., preferably in the range of from 125 to 175° C., more preferably in the range of from 130 to 160° C., more preferably in the range of from 135 to 155° C. more preferably in the range of from 140 to 150° C.
49. The process of any one of embodiments 22 to 48, wherein the water treatment according to (iii) is carried out under autogenous pressure, preferably in an autoclave.
50. The process of any one of embodiments 22 to 49, wherein the water treatment according to (iii) is carried out for 6 to 10 h, preferably for 7 to 9 h, more preferably for 7.5 to 8.5 h.
51. The process of any one of embodiments 22 to 50, wherein in the mixture prepared in (iii), the weight ratio of the zeolitic material relative to the water is in the range of from 1:1 to 1:10, preferably in the range of from 1:3 to 1:7, more preferably in the range of from 1:4 to 1:6.
52. The process of any one of embodiments 22 to 51, wherein after (iii) and prior to (iv), the water-treated precursor of the molding is separated from the mixture obtained from (iii), wherein separating preferably comprises subjecting the mixture obtained from (iii) to filtration or centrifugation, wherein more preferably, separating further comprises washing the water-treated precursor of the molding at least once with a liquid solvent system, wherein the liquid solvent system preferably comprises one or more of water, an alcohol, and a mixture of two or more thereof, wherein the water-treated precursor of the molding is more preferably washed with water.
53. The process of any one of embodiments 22 to 52, preferably 52, wherein after (iii) and prior to (iv), the preferably separated water-treated precursor of the molding is dried in a gas atmosphere, wherein drying is preferably carried out at a temperature of the gas atmosphere in the range of from 80 to 160° C., more preferably in the range of from 100 to 140° C., more preferably in the range of from 110 to 130° C., wherein the gas atmosphere preferably comprises nitrogen, oxygen, or a mixture thereof, wherein the gas atmosphere is more preferably oxygen, air or lean air.
54. The process of any one of embodiments 22 to 53, wherein calcining in (iv) is carried out at a temperature of the gas atmosphere in the range of from 400 to 490° C., preferably in the range of from 420 to 470° C., more preferably in the range of from 440 to 460° C.
55. The process of any one of embodiments 22 to 54, wherein the gas atmosphere according to (iv) comprises nitrogen, oxygen, or a mixture thereof, wherein the gas atmosphere is preferably oxygen, air or lean air.
56. The process of any one of embodiments 22 to 55, comprising preparing the zeolitic material according to (i) by a method comprising, preferably consisting of
   (a) providing a zeolitic material having framework type MFI, wherein from 98 to 100 weight-% of the zeolitic material consist of Ti, Si, O, and H, and wherein the zeolitic material having framework type MFI exhibits a type I nitrogen adsorption/desorption isotherm determined as described in Reference Example 1;
   (b) preparing an aqueous mixture comprising the zeolitic material provided in (i) and a zeolite framework type MFI structure directing agent;
   (c) subjecting the mixture obtained from (b) to hydrothermal conditions under autogenous pressure, preferably in an autoclave, obtaining a suspension comprising a precursor of the zeolitic material having framework type MFI according to (i), and separating said precursor from the suspension;
   (d) optionally calcining the precursor in a gas atmosphere;
   (e) subjecting the precursor obtained from (c) or (d) to an acid treatment and preferably drying the acid-treated precursor;
   (f) calcining the acid-treated precursor, obtaining the zeolitic material having framework type MFI according to (i).
57. A molding comprising a zeolitic material having framework type MFI and a silica binder, obtainable or obtained by a process according to any one of embodiment 22 to 56.
58. Use of a molding according to any one of embodiments 1 to 21 or according to embodiment 57 as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component, more preferably as an oxidation catalyst or as an oxidation catalyst component, more preferably as an epoxidation catalyst or as an epoxidation catalyst component, more preferably as an epoxidation catalyst.
59. The use of embodiment 58 for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene, more preferably for the epoxidation of propene with hydrogen peroxide as oxidizing agent, more preferably for the epoxidation of propene with hydrogen peroxide as oxidizing agent in a solvent comprising an alcohol, preferably methanol.
60. A process for oxidizing an organic compound comprising bringing the organic compound in contact with a catalyst comprising a molding according to any one of embodiments 1 to 21 or according to embodiment 57, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.
61. The process of embodiment 60, wherein hydrogen peroxide is used as oxidizing agent, wherein the oxidation reaction is preferably carried out in a solvent, more preferably in a solvent comprising an alcohol, preferably methanol.
62. A process for preparing propylene oxide comprising reacting propene with hydrogen peroxide in methanolic solution in the presence of a catalyst comprising a molding according to any one of embodiments 1 to 21 or according to embodiment 57 to obtain propylene oxide.
63. The process of embodiment 62, wherein a reaction feed comprising propene, methanol and hydrogen peroxide is introduced into a reactor, said reaction feed containing potassium cations (K⁺) in an amount of from 100 to 160 micromol, relative to 1 mol hydrogen peroxide contained in the reaction feed, and further containing phosphorus (P) in the form of anions of at least one phosphorus oxyacid.
64. The process of embodiment 63, wherein the reaction feed contains K⁺ in an amount of from 100 to 155 micromol, preferably of from 120 to 150 micromol, relative to 1 mol hydrogen peroxide contained the reaction feed.
65. The process of embodiment 63 or 64, wherein in the reaction feed, the molar ratio of K⁺ relative to P is in the range of from 1.5:1 to 2.5:1, preferably of from 1.9:1 to 2.1:1.
66. The process of any one of embodiments 63 to 65, wherein the reaction feed is obtained from a hydrogen peroxide feed, a methanol feed, and a propene feed.
67. The process of embodiment 66, wherein the hydrogen peroxide feed contains K⁺ in an amount of less than 110 micromol, preferably less than 70 micromol, more preferably less than 30 micromol, in particular less than 5 micromol, relative to 1 mol hydrogen peroxide contained in the hydrogen peroxide feed.
68. The process of embodiment 67, wherein at least one solution containing K⁺ and P in the form of anions of at least one phosphorus oxyacid is added to the hydrogen peroxide feed or to the propene feed or to the methanol feed or a mixed feed of two or three thereof, in such an amount that the reaction feed contains K⁺, and P in the form of anions of at least one phosphorus oxyacid in amounts as defined in any one of embodiments 63 to 65.
69. The process of embodiment 68, wherein the at least one solution is an aqueous solution of dipotassium hydrogen phosphate.
70. The process of any one of embodiments 66 to 69, wherein the hydrogen peroxide feed is an aqueous or a methanolic or an aqueous/methanolic, preferably an aqueous hydrogen peroxide feed, containing hydrogen peroxide preferably in an amount of from 25 to 75 wt.-%, more preferably of from 30 to 50 wt.-%.
71. The process of any one of embodiments 66 to 70, wherein the propene feed additionally contains propane wherein the volume ratio of propene to propane is preferably in the range of from 99.99:0.01 to 95:5.
72. The process of any one of embodiments 63 to 71, wherein the reaction feed when introduced into the reactor consists of one liquid phase.
73. The process of any one of embodiments 62 to 72, wherein the pressure under which the reaction of propene with hydrogen peroxide in methanolic solution in the presence of the catalyst comprising the molding is carried out in the reactor is at least 10 bar(abs), preferably at least 15 bar(abs), more preferably at least 20 bar(abs), more preferably in the range of from 20 to 40 bar(abs).
74. The process of any one of embodiments 62 to 73, wherein the reaction mixture in the reactor is externally and/or internally cooled such that the maximum temperature of the reaction mixture in the reactor is in the range of from 30 to 70° C.
75. The process any one of embodiments 62 to 74, wherein the reaction of propene with hydrogen peroxide in methanolic solution in the presence of the catalyst comprising the molding is carried out by a method comprising
   (i) reacting propene with hydrogen peroxide in methanolic solution in the presence of a catalyst comprising the molding in at least one reactor R1 which is preferably operated in isothermal mode, wherein a reaction feed comprising propene, methanol and hydrogen peroxide is introduced into R1, said reaction feed containing potassium cations (K⁺) in an amount of from 100 to 160 micromol, relative to 1 mol hydrogen peroxide contained in the reaction feed, and further containing phosphorus (P) in the form of anions of at least one phosphorus oxyacid;
   (ii) separating a stream containing non-reacted hydrogen peroxide from the reaction mixture obtained from (i) and removed from R1, said separating preferably being carried out by distillation in at least 1, preferably 1 distillation column K1;
   (iii) mixing the stream containing non-reacted hydrogen peroxide with a propene stream, passing the mixed stream into at least 1, preferably 1 reactor R2 containing a catalyst comprising the molding and preferably being operated in adiabatic mode, and reacting propene with hydrogen peroxide in R2.

According to a further aspect, the present invention relates to a zeolitic material having framework type MFI wherein from 98 to 100 weight-% of the zeolitic material consist of Ti, Si, O, P, and H, and wherein the zeolitic material having framework type MFI exhibits a type IV nitrogen adsorption/desorption isotherm determined as described in Reference Example 1. Therefore, the present invention is further illustrated by the following set of embodiments and combinations of embodiments resulting from the dependencies and back-references as indicated. In particular, it is noted that in each instance where a range of embodiments is mentioned, for example in the context of a term such as “The zeolitic material of any one of embodiments 1′ to 4′”, every embodiment in this range is meant to be explicitly disclosed for the skilled person, i.e. the wording of this term is to be understood by the skilled person as being synonymous to “The molding of any one of embodiments 1′, 2′, 3′, and 4′”.
1′. A P-containing zeolitic material having framework type MFI wherein from 98 to 100 weight-% of the zeolitic material consist of Ti, Si, O, P, and H, and wherein the zeolitic material having framework type MFI exhibits a type IV nitrogen adsorption/desorption isotherm determined as described in Reference Example 1.
2′. The zeolitic material of embodiment 1′, wherein from 99 to 100 weight-%, preferably from 99.5 to 100 weight-%, more preferably from 99.9 to 100 weight-%, of the zeolitic material consist of Ti, Si, O, P and H.
3′. The zeolitic material of embodiment 1′ or 2′, having a Ti content in the range of from 0.5 to 3.0 weight-%, preferably in the range of from 1.0 to 2.0 weight-%, more preferably in the range of from 1.2 to 1.8 weight-%, calculated as elemental Ti and based on the weight of zeolitic material.
4′. The zeolitic material of any one of embodiments 1′ to 3′, having a P content in the range of from 0.5 to 6.0 weight-%, preferably in the range of from 1 to 5 weight-%, more preferably in the range of from 2 to 4 weight-%, calculated as elemental P and based on the weight of zeolitic material.
5′. The zeolitic material of any one of embodiments 1′ to 4′, having a BET specific surface area of at least 250 m²/g, preferably in the range of from 250 to 500 m²/g, more preferably in the range of from 300 to 450 m²/g, determined as described in Reference Example 6.
6′. The zeolitic material of any one of embodiments 1′ to 5′, exhibiting a ²⁹Si direct excitation solid-state NMR spectrum comprising three resonances having areas of (74.5±3x) %, (23.5±2x) %, and (2.0±x) %, wherein x is 2, preferably 1, more preferably 0.5.
7′. The zeolitic material of any one of embodiments 1′ to 6′, exhibiting a ²⁹Si cross-polarization solid-state NMR spectrum comprising a main resonance with a peak in the range of from −108 to −120 ppm having an area I₀, and preferably having a minor resonance with a peak in the range of from −95 to −108 ppm having an area I₁, wherein the ratio I₀:I₁ is preferably in the range of from 0.7:1 to 1.3:1, more preferably in the range of from 0.85:1 to 1.15:1, more preferably in the range of from 0.9:1 to 1.1:1.
8′. The zeolitic material of any one of embodiments 1′ to 7′, exhibiting a ³¹P direct excitation solid-state NMR spectrum comprising five resonances with peaks at (−2±0.2) ppm, (−11±0.2) ppm, (−23±0.2) ppm, (−31±0.2) ppm, and (−44±0.2) ppm, and optionally further resonances, wherein in the spectrum, the ratio of the integral I₀ over the range of from +7 to −23 ppm relative to the integral I₁ over the range of from −23 to −53 ppm is preferably in the range of from 0.5:1 to 1.1:1, more preferably in the range of from 0.65:1 to 0.95:1, more preferably in the range of from 0.7:1 to 0.9:1.
9′. The zeolitic material of any one of embodiments 1′ to 8′, exhibiting a ³¹P cross-polarization solid-state NMR spectrum comprising five resonances with peaks at (−2±0.2) ppm, (−11±0.2) ppm, (−23±0.2) ppm, (−31±0.2) ppm, and (−44±0.2) ppm, and optionally further resonances, wherein in the spectrum, the ratio of the integral I₀ over the range of from +7 to −23 ppm relative to the integral I₁ over the range of from −23 to −53 ppm is preferably in the range of from 0.5:1 to 1.1:1, more preferably in the range of from 0.65:1 to 0.95:1, more preferably in the range of from 0.7:1 to 0.9:1.
10′. A process for preparing the P-containing zeolitic material according to any one of embodiments 1′ to 9′, comprising
   (i′) preparing an aqueous mixture comprising a zeolitic material having framework type MFI wherein from 98 to 100 weight-% of the zeolitic material consist of Ti, Si, O, and H, and wherein the zeolitic material having framework type MFI exhibits a type IV nitrogen adsorption/desorption isotherm determined as described in Reference Example 1, the mixture further comprising a source of P;
   (ii′) drying the mixture prepared in (i′) in a gas atmosphere, obtaining a precursor of the P-containing zeolitic material;
   (iii′) calcining the dried precursor of the P-containing zeolitic material, obtaining the P-containing zeolitic material.
11.′ The process of embodiment 10′, wherein the zeolitic material according to (i′) comprises hollow cavities having a diameter of greater than 5.5 Angstrom, preferably in the range of greater than 5.5 Angstrom to smaller than the size of the crystallite of the zeolitic material, determined via TEM as described in Reference Example 3.
12′. The process of embodiment 10′ or 11′, wherein from 99 to 100 weight-%, preferably from 99.5 to 100 weight-%, more preferably from 99.9 to 100 weight-%, of the zeolitic material according to (i′) consist of Ti, Si, O, and H.
13′. The process of any one of embodiments 10′ to 12′, wherein the zeolitic material according to (i′) has a sodium content, calculated as Na₂O, in the range of from 0 to 0.1 weight-%, preferably in the range of from 0 to 0.07 weight-%, more preferably in the range of from 0 to 0.05 weight-%, based on the weight of the zeolitic material.
14′. The process of any one of embodiments 10′ to 13′, wherein the zeolitic material according to (i′) has an iron content, calculated as Fe₂O₃, in the range of from 0 to 0.1 weight-%, preferably in the range of from 0 to 0.07 weight-%, more preferably in the range of from 0 to 0.05 weight-%, based on the weight of the zeolitic material.
15′. The process of any one of embodiments 10′ to 14′, wherein the zeolitic material according to (i′) has a Ti content in the range of from 1.3 to 2.1 weight-%, preferably in the range of from 1.5 to 1.9 weight-%, more preferably in the range of from 1.6 to 1.8 weight-%, calculated as elemental Ti and based on the weight of zeolitic material.
16′. The process of any one of embodiments 10′ to 15′, wherein the source of P comprises one or more phosphorus oxoacids, preferably one or more of hypophosphoric acid, hypophosphorus acid, phosphoric acid, phosphorous acid, pyrophosphoric acid, and triphosphoric acid, more preferably phosphoric acid (H₃PO₄).
17′. The process of any one of embodiments 10′ to 16′, wherein drying according to (ii′) is carried out at a temperature of the gas atmosphere in the range of from 50 to 150° C., preferably in the range of from 70 to 140° C., more preferably in the range of from 90 to 130° C., wherein the gas atmosphere preferably comprises nitrogen, oxygen, or a mixture thereof, wherein the gas atmosphere is more preferably oxygen, air, or lean air.
18′. The process of any one of embodiments 10′ to 17′, wherein calcining according to (iii′) is carried out at a temperature of the gas atmosphere in the range of from 400 to 600° C., preferably in the range of from 450 to 550° C., more preferably in the range of from 475 to 525° C., wherein the gas atmosphere preferably comprises nitrogen, oxygen, or a mixture thereof, wherein the gas atmosphere is more preferably oxygen, air, or lean air.
19′. A P-containing zeolitic material, preferably the P-containing zeolitic material according to any one of embodiments 1′ to 9′, obtainable or obtained by a process according to any one of embodiments 10′ to 18′.
20′. A molding comprising the zeolitic material according to any one of embodiments 1′ to 9′ and an oxidic binder, said molding optionally being obtainable according to a process as described in any one of embodiments 22 to 56 wherein instead of the zeolitic material according to (i), the zeolitic material according to any one of embodiments 1′ to 9′ is employed.
21′. Use of a zeolitic material according to any one of embodiments 1′ to 9′ or according to embodiment 19′ or of a molding according to embodiment 20′ as an adsorbent, as an absorbent, as a catalyst or as a catalyst component.

The present invention is further illustrated by the following examples and reference examples.

### Reference Example 1: Determination of N₂ Adsorption/Desorption Isotherm

The nitrogen adsorption/desorption isotherm was determined at 77 K according to the method disclosed in DIN 66131.

### Reference Example 2: Determination of the Total Pore Volume

The total pore volume was determined via intrusion mercury porosimetry according to DIN 66133.

### Reference Example 3: Determination of the Size of the Hollow Cavities of the Zeolitic Material

The size of the hollow cavities of the zeolitic material was determined via TEM. Samples for Transmission Electron Microscopy (TEM) were prepared on ultra-thin carbon TEM carriers. The powder was therefore dispersed in ethanol. One drop of the dispersion was applied between two glass objective slides and gently dispersed. The TEM carrier film was subsequently dipped on the resulting thin film. The samples were imaged by TEM using a Tecnai Osiris machine (FEI Company, Hillsboro, USA) operated at 200 keV under bright-field as well as high-angle annular dark-field scanning TEM (HAADF-STEM) conditions. Chemical composition maps were acquired by energy-dispersive x-ray spectroscopy (EDXS). Images and elemental maps were evaluated using the iTEM (Olympus, Tokyo, Japan, version: 5.2.3554) as well as the Esprit (Bruker, Billerica, USA, version 1.9) software packages.

### Reference Example 4: Determination of the Hardness

The crush strength as referred to in the context of the present invention is to be understood as having been determined via a crush strength test machine Z2.5/TS1S, supplier Zwick GmbH & Co., D-89079 Ulm, Germany. As to fundamentals of this machine and its operation, reference is made to the respective instructions handbook “Register 1: Betriebsanleitung/Sicherheitshandbuch für die Material-Prüfmaschine Z2.5/TS1S”, version 1.5, December 2001 by Zwick GmbH & Co. Technische Dokumentation, August-Nagel-Strasse 11, D-89079 Ulm, Germany. The machine was equipped with a fixed horizontal table on which the strand was positioned. A plunger having a diameter of 3 mm which was freely movable in vertical direction actuated the strand against the fixed table. The apparatus was operated with a preliminary force of 0.5 N, a shear rate under preliminary force of 10 mm/min and a subsequent testing rate of 1.6 mm/min. The vertically movable plunger was connected to a load cell for force pick-up and, during the measurement, moved toward the fixed turntable on which the molding (strand) to be investigated is positioned, thus actuating the strand against the table. The plunger was applied to the strands perpendicularly to their longitudinal axis. With said machine, a given strand as described below was subjected to an increasing force via a plunger until the strand was crushed. The force at which the strand crushes is referred to as the crushing strength of the strand. Controlling the experiment was carried out by means of a computer which registered and evaluated the results of the measurements. The values obtained are the mean value of the measurements for 10 strands in each case.

### Reference Example 5: Determination of Dv10, Dv50, and Dv90 Values

Sample Preparation:

1.0 g of the sample is suspended in 100 g deionized water and stirred for 1 min. Apparatus and respective parameters used:
Mastersizer S long bed version 2.15, ser. No. 33544-325; supplier: Malvern Instruments GmbH, Herrenberg, Germany
focal width: 300RF mm
beam length: 10.00 mm
module: MS17
shadowing: 16.9%
dispersion model:3$$D
analysis model: polydisperse
correction: none

### Reference Example 6: Determination of the BET Specific Surface Area

The BET specific surface area was determined via nitrogen physisorption at 77 K according to the method disclosed in DIN 66131. The N₂ sorption isotherms at the temperature of liquid nitrogen were measured using Micrometrics ASAP 2020M and Tristar system for determining the BET specific surface area.

### Reference Example 7: Determination of the Langmuir Surface Area

The Langmuir surface area was determined via nitrogen physisorption at 77 K according to the method disclosed in DIN 66131.

### Reference Example 8: X-Ray Powder Diffraction and Determination of the Crystallinity

Powder X-ray diffraction (PXRD) data was collected using a diffractometer (D8 Advance Series II, Bruker AXS GmbH) equipped with a LYNXEYE detector operated with a Copper anode X-ray tube running at 40 kV and 40 mA. The geometry was Bragg-Brentano, and air scattering was reduced using an air scatter shield.

Computing crystallinity: The crystallinity of the samples was determined using the software DIFFRAC.EVA provided by Bruker AXS GmbH, Karlsruhe. The method is described on page 121 of the user manual. The default parameters for the calculation were used.

Computing phase composition: The phase composition was computed against the raw data using the modelling software DIFFRAC.TOPAS provided by Bruker AXS GmbH, Karlsruhe. The crystal structures of the identified phases, instrumental parameters as well the crystallite size of the individual phases were used to simulate the diffraction pattern. This was fit against the data in addition to a function modelling the background intensities.

Data collection: The samples were homogenized in a mortar and then pressed into a standard flat sample holder provided by Bruker AXS GmbH for Bragg-Brentano geometry data collection. The flat surface was achieved using a glass plate to compress and flatten the sample powder. The data was collected from the angular range 2 to 70° 2Theta with a step size of 0.02° 2Theta, while the variable divergence slit was set to an angle of 0.1°. The crystalline content describes the intensity of the crystalline signal to the total scattered intensity. (User Manual for DIFFRAC.EVA, Bruker AXS GmbH, Karlsruhe.)

### Reference Example 9: Determination of ²⁹Si Solid State NMR

The ²⁹Si solid state NMR direct excitation was carried with 5 μs 90°-pulse, free induction decay (FID) acquisition of 30 ms, heteronuclear radiofrequency decoupling (HPPD) at 50 kHz ¹H nutation frequency, averaging of at least 128 scans with a recycle delay of 120s. The spectra was referenced relative to the unified scale according to Pure Appl. Chem., Vol. 80, No. 1, pp. 59-84, 2008, ²⁹Si frequencies are given relative to ²⁹Si of Me₄Si (Tetramethylsilane, TMS) in CDCl₃ (1% volume fraction) with a frequency ratio of 19.867187% on the unified scale, in ppm. Direct spectrum integrations were performed using Bruker Topspin 3.

### Reference Example 10: Determination of the Propylene Oxide Activity and the Pressure Drop Rate (PO Test)

In the PO test, a preliminary test procedure to assess the possible suitability of the moldings as catalyst for the epoxidation of propene, the moldings were tested in a glass autoclave by reaction of propene with an aqueous hydrogen peroxide solution (30 weight-%) to yield propylene oxide. In particular, 0.5 g of the molding were introduced together with 45 mL of methanol in a glass autoclave, which was cooled to −25° C. 20 mL of liquid propene were pressed into the glass autoclave and the glass autoclave was heated to 0° C. At this temperature, 18 g of an aqueous hydrogen peroxide solution (30 weight-% in water) were introduced into the glass autoclave. After a reaction time of 5 h at 0° C., the mixture was heated to room temperature and the liquid phase was analyzed by gas chromatography with respect to its propylene oxide content. The propylene oxide content of the liquid phase (in weight-%) is the result of the PO test, i.e. the propylene oxide activity of the molding.

The pressure drop rate was determined following the pressure progression during the PO test described above. The pressure progression was recorded using a S-11 transmitter (from Wika Alexander Wiegand SE & Co. KG), which was positioned in the pressure line of the autoclave, and a graphic plotter Buddeberg 6100A. The respectively obtained data were read out and depicted in a pressure progression curve. The pressure drop rate (PDR) was determined according to the following equation:

PDR=[p(max)−p(min)]/delta t
with
PDR/(bar/min)=pressure drop rate
p(max)/bar=maximum pressure at the start of the reaction
p(min)/bar=minimum pressure observed during the reaction
delta t/min=time difference from the start of the reaction to the point in time where p(min) was observed

### Reference Example 11: Determination of the Propylene Epoxidation Catalytic Performance

In a continuous epoxidation reaction setup, a vertically arranged tubular reactor (length: 1.4 m, outer diameter 10 mm, internal diameter: 7 mm) equipped with a jacket for thermostatization was charged with 15 g of the moldings in the form of strands as described in the respective examples below. The remaining reactor volume was filled with inert material (steatite spheres, 2 mm in diameter) to a height of about 5 cm at the lower end of the reactor and the remainder at the top end of the reactor. Through the reactor, the starting materials were passed with the following flow rates: methanol (49 g/h); hydrogen peroxide (9 g/h; employed as aqueous hydrogen peroxide solution with a hydrogen peroxide content of 40 weight-%); propylene (7 g/h; polymer grade). Via the cooling medium passed through the cooling jacket, the temperature of the reaction mixture was adjusted so that the hydrogen peroxide conversion, determined on the basis of the reaction mixture leaving the reactor, was essentially constant at 90%. The pressure within the reactor was held constant at 20 bar(abs), and the reaction mixture—apart from the fixed-bed catalyst—consisted of one single liquid phase.

The reactor effluent stream downstream the pressure control valve was collected, weighed and analyzed. Organic components were analyzed in two separate gas-chromatographs. The hydrogen peroxide content was determined colorimetrically using the titanyl sulfate method. The selectivity for propylene oxide given was determined relative to propene and hydrogen peroxide), and was calculated as 100 times the ratio of moles of propylene oxide in the effluent stream divided by the moles of propene or hydrogen peroxide in the feed.

### Reference Example 12: Preparation of a Zeolitic Material Having Framework Type MFI (Hollow TS-1 (HTS-1))

### Reference Example 12.1: Preparation of a Zeolitic Material Having Framework Type MFI Wherein from 98 to 100 Weight-% of the Zeolitic Material Consist of Ti, Si, O, and H (Titanium Silicalite-1 (TS-1))

A titanium silicalite-1 (TS-1) powder was prepared according to the following recipe: TEOS (tetraethyl orthosilicate) (300 kg) were loaded into a stirred tank reactor at room temperature and stirring (100 r.p.m.) was started. In a second vessel, 60 kg TEOS and 13.5 kg TEOT (tetraethyl orthotitanate) were first mixed and then added to the TEOS in the first vessel. Subsequently, another 360 kg TEOS were added to the mixture in the first vessel. Then, the content of the first vessel was stirred for 10 min before 950 g TPAOH (tetrapropylammonium hydroxide) were added. Stirring was continued for 60 min. Ethanol released by hydrolysis was separated by distillation at a bottoms temperature of 95° C. 300 kg water were then added to the content of the first vessel, and water in an amount equivalent to the amount of distillate was further added. The obtained mixture was stirred for 1 h. Crystallization was performed at 175° C. within 12 h at autogenous pressure. The obtained titanium silicalite-1 crystals were separated, dried, and calcined at a temperature of 500° C. in air for 6 h.

### Reference Example 12.2: Preparation of Hollow TS-1 (HTS-1)

1072 g deionized water were provided in a beaker. Then, 424 g tetrapropylammonium hydroxide (as an aqueous solution comprising 40 weight-% tetrapropylammonium hydroxide) were added under stirring. Subsequently, 200 g of a TS-1 powder prepared according to Reference Example 12.1 were added. This mixture was homogenized for 30 min. The mixture was then transferred in an autoclave. The mixture was hydrothermally treated at 170° C. for 24 hours. The resulting suspension was filtrated, and the solid residue obtained was washed with deionized water. The resulting solid was dried overnight at room temperature. The yield was 150 g.

3000 g of aqueous nitric acid (10 weight-% HNO₃ in water) were provided in a glass beaker. Under stirring, 150 g of the dried solid were added thereto. The resulting suspension—while being stirred at 250 rpm—was refluxed at 100° C. for 1 hour. For work-up, the suspension was filtrated, and the solid residue was washed with deionized water. The resulting solid was dried in air and subsequently calcined in air in an oven according to the following procedure:

1. heating up to 120° C. within 1 hour

2. drying at 120° C. for 4 hours

3. heating up to 500° C. within 190 min

4. calcining at 500° C. for 5 hours.

The solid was then grinded. The yield was 121 g. The resulting powder had a TOC of less than 0.1 g/100 g, a Si content of 44 g/100 g, and a Ti content of 1.7 g/100 g, showed a water adsorption/desorption at 85% relative humidity of less than 8.5, a BET specific surface area of 453 m²/g, and a Langmuir surface area of 601 m²/g, each determined as described hereinabove. As determined by X-ray diffraction analysis, the sample essentially consisted of HTS-1 (1 weight-% crystalline anatase and 99 weight-% of crystalline HTS-1).

### Example 1: Preparing a Molding According to the Invention

### Example 1.1: Shaping of an HTS-1 Powder

50 g of the zeolitic material of Reference Example 12.2 and 2 g Walocel™ (Walocel MW 15000 GB, Wolff Cellulosics GmbH & Co. KG, Germany) were provided in a kneader and kneaded for 5 minutes. Then, 50.4 g of an aqueous dispersion comprising 25 weight-% polystyrene and 25 weight-% silica (colloidal Ludox® AS 40 was used as basis for the dispersion) were added. After 10 minutes, 0.67 g polyethylene oxide (PEO, Union Carbide, PolyOX Coagulant) were added and the mixture was kneaded. After further 10 minutes, 41.65 g of a colloidal silica (Ludox® AS 40) were added. Subsequently, the addition of de-ionized water was started in portions of 10 ml every 10 minutes to result in a total addition of water of 100 mL. The total kneading time was 45 minutes. After completion of the water addition, the kneaded mass was subjected to shaping. For shaping, the kneaded mass was extruded at a pressure of 150 bar(abs) to give strands with a circular cross-section having a diameter of 1.7 mm. The strands were then dried and calcined in air according to the following program:

1. heating within 60 minutes to a temperature of 120° C.;

2. keeping the temperature of 120° C. for 4 h;

3. heating within 185 minutes to a temperature of 490° C.;

4. keeping the temperature of 490° C. for 5 h.

The yield was 55 g.

### Example 1.2: Water Treatment of Shaped HTS-1

36 g of the strands prepared according to Example 1.1 were mixed in four portions of each 9 g with 180 g deionized water per portion. The resulting mixtures were heated to a temperature of 145° C. for 8 h in an autoclave. Thereafter, the obtained water-treated strands were separated and was sieved over a 0.8 mm sieve. The obtained strands were then washed with deionized water and pre-dried in a stream of nitrogen at ambient temperature. The washed and pre-dried strands were subsequently dried and calcined in air according to the following program:

1. heating within 60 minutes up to 120° C.;

2. keeping the temperature of 120° C. for 4 h;

3. heating within 165 minutes up to 450° C.;

4. keeping the temperature of 450° C. for 2 h.

The yield was 36.2 g. The resulting material had a TOC of less 0.1 g/100 g, a Si content of 45 g/100 g, and a Ti content of 1.3 g/100 g, each determined as described hereinabove. The hardness of the strands determined as described hereinabove was 4.3 N, and the pore volume determined as described hereinabove was 0.82 ml/g.

### Example 2: Preparing a Molding According to the Invention

### Example 2.1: Shaping of an HTS-1 Powder

79 g of a commercially available HTS-1 powder (Titanium Silicalite RT-03 of Zhejiang TWRD New Material Co., Ltd., CN) and 3 g Walocel™ (Walocel MW 15000 GB, Wolff Cellulosics GmbH & Co. KG, Germany) were provided in a kneader and kneaded for 5 minutes. Then, 75.5 g of an aqueous dispersion comprising 25 weight-% polystyrene and 25 weight-% colloidal silica (Ludox® AS 40 was used as basis for the dispersion) were added. After 10 minutes, 1.0 g polyethylene oxide (PEO, Union Carbide, PolyOX Coagulant) was added and the mixture was kneaded. After further 10 minutes, 70 g of a colloidal silica (Ludox® AS 40) were added. The mass was kneaded for further 10 minutes. The total kneading time was 35 minutes. The kneaded mass was extruded at a pressure of 120 bar(abs) to give strands having a circular cross-section with a diameter of 1.9 mm. The extruded strands were then dried and calcined in air according to the following program:

1. heating within 60 minutes up to a temperature of 120° C.;

2. keeping the temperature of 120° C. for 4 h;

3. heating within 185 minutes to a temperature of 490° C.;

4. keeping the temperature of 490° C. for 5 h.

The yield was 90 g. The resulting material had a TOC of less than 0.1 g/100 g, a Si content of 45 g/100 g, and a Ti content of 1.2 g/100 g, each determined as described hereinabove. The hardness of the strands determined as described hereinabove was 0.85 N, and the pore volume determined as described hereinabove was 0.76 ml/g. The crystallinity determined as described hereinabove was 78%.

### Example 2.2: Water Treatment of Shaped HTS-1

36 g of the material prepared according to example 2.1 were mixed in four portions of each 9 g with 180 g deionized water per portion. The resulting mixtures were heated at a temperature of 145° C. for 8 h in an autoclave. Thereafter, the strands were sieved over a 0.8 mm sieve. The strands were washed with deionized water and pre-dried in a stream of nitrogen at ambient temperature. The washed and pre-dried strands were subsequently dried and calcined in air according to the following program:

1. heating within 60 minutes up to 120° C.;

2. keeping the temperature of 120° C. for 4 h;

3. heating within 165 minutes up to 450° C.;

4. keeping the temperature of 450° C. for 2 h.

The yield was 34.9 g. The resulting material had a TOC of less than 0.1 g/100 g, a Si content of 45 g/100 g, and a Ti content of 1.3 g/100 g, showed a BET specific surface area of 345 m²/g, each determined as described hereinabove. The hardness of the strands determined as described hereinabove was 8.4 N, and the pore volume determined as described hereinabove was 1.0 ml/g.

### Comparative Examples: Preparing a Molding According to the Prior Art

The following comparative examples were prepared allowing a comparison of the moldings of the prior art, comprising HTS-1, with the moldings according to the present invention in particular in view of the catalytic performance in an epoxidation reaction. In view of cited prior art Xu et al. mentioned hereinabove, moldings according to Comparative Example 1 were prepared wherein, in particular, Sesbania cannabina Pers. powder was used. The moldings of Comparative Example 2 were prepared based on the prior art Liu et al. wherein sepiolite is employed in the preparation process. Therefore, the comparative examples have been carried out particularly taking into consideration the prior art.

### Comparative Example 1: Shaping of an HTS-1 Powder

Based on the general teaching of J. Xu et al.: “Effect of triethylamine treatment of titanium silicalite-1 on propylene epoxidation”, 150 g of commercially available HTS-1 powder (Titanium Silicalite RT-03 of Zhejiang TWRD New Material Co., Ltd., CN) and 6 g of Sesbania cannabina Pers. powder were provided in a kneader and kneaded for 5 minutes. Then, 75 g of c collodal silica (Ludox® AS 40) were added. The mass was kneaded for further 10 minutes. Then, 40 mL of water were added and the mixture was further kneaded for 5 minutes. After that, further 20 mL of water were added and the mixture kneaded for further 10 minutes. The total kneading time was 30 minutes. A portion of the kneaded mass was extruded at a pressure of 150 bar(abs) to give strands having a circular cross-section with a diameter of 1.7 mm. The rest of the kneaded mass was extruded at a pressure of 205 bar(abs). Subsequently, the extruded strands were then dried and calcined in air according to the following program:

1. heating within 60 minutes up to a temperature of 120° C.;

2. keeping the temperature of 120° C. for 6 h;

3. heating within 165 minutes to a temperature of 550° C.;

4. keeping the temperature of 550° C. for 6 h.

The yield was 63.2 g. The resulting material had a Ti content of 1.3 g/100 g, a BET specific surface area of 369 m²/g, and a Langmuir surface area of 495 m²/g, each determined as described hereinabove. The hardness of the strands determined as described hereinabove was 5.2 N, and the pore volume determined as described hereinabove was 0.45 ml/g. The crystallinity determined as described hereinabove was 79%.

### Comparative Example 2: Shaping of an HTS-1 Powder

Based on the general teaching of M. Liu et al. “Highly Selective Epoxidation of Propylene in a Low-Pressure Continuous Slurry Reactor and the Regeneration of Catalyst, 54 g of commercially available zeolitic HTS-1 (Titanium Silicalite RT-03 of Zhejiang TWRD New Material Co., Ltd., CN), 36 g sepiolite with a content of approx. 13 weight-% Mg, CAS 63800-37-3, Aldrich) and 10 g methylcellulose (Walocel MW 15000 GB, Wolff Cellulosics GmbH & Co. KG, Germany) were provided in a kneader and kneaded for 5 minutes. Then, 40 mL of water were added and the mixture was further kneaded for 15 minutes. After that, further 25 mL of water were added and the mixture kneaded for further 10 minutes. The total kneading time was 30 minutes. The kneaded mass was extruded at a pressure of 150 bar(abs) to give strands having a circular cross-section with a diameter of 1.7 mm. Subsequently, the extruded strands were dried and calcined in air according to the following program:

1. heating within 40 minutes up to a temperature of 80° C.;

2. keeping the temperature of 80° C. for 6 h;

3. heating within 265 minutes to a temperature of 650° C.;

4. keeping the temperature of 650° C. for 6 h.

The yield was 77.9 g. The resulting material had a TOC of less than 0.1 g/100 g, a Si content of 40 g/100 g, a Mg content of 5.8 g/100 g, and a Ti content of 1.1 g/100 g, showed a BET specific surface area of 314 m²/g, each determined as described hereinabove. The hardness of the strands determined as described hereinabove was 10 N, and the pore volume determined as described hereinabove was 0.5 ml/g.

For a better overview, the pore volumes of the moldings of the present invention comprising HTS-1 and the pore volumes of the moldings of the prior art comprising HTS-1 are shown in the the Table 1 below:

TABLE 1
Pore volumes determined according to Reference Example 2

| Molding according to # | pore volume/ml/g |
|---|---|
| Example 1.2 | 0.82 |
| Example 2.2 | 1.00 |
| Comparative Example 1 | 0.45 |
| Comparative Example 2 | 0.50 |

### Comparative Example 3: Shaping of a TS-1 Powder

Examples 1.1 and 1.2 were repeated wherein in Example 1.1, instead of the HTS-1 powder according to Reference Example 12.2, the (non-hollow) TS-1 powder according to Reference example 12.1 was employed as the zeolitic material. The respectively obtained strands comprising TS-1 were further processed (water-treated) as described in Example 1.2.

### Example 3: Catalytic Testings

### Example 3.1: Preliminary Test—PO Test

Moldings of the examples were preliminarily tested with respect to their general suitability as expoxidation catalysts according to the PO test as described in Reference Example 10. The respective resulting values of the propylene oxide activity are shown in Table 2 below.

TABLE 2
Results for catalytic testing according to Reference Example 10

| Molding according to # | propylene oxide activity/% |
|---|---|
| Example 1.2 | 4.6 |
| Example 2.2 | 5.3 |

Obviously, the moldings according to the present invention exhibit a very good propylene oxide activity according to the PO test and are promising candidates for catalysts in industrial continuous epoxidation reactions.

### Example 3.2: Catalytic Characteristics of the Moldings in a Continuous Epoxidation Reaction

The characteristics of moldings of the present invention were compared with moldings of the prior art in a continuous epoxidation reaction as described in Reference Example 11. After a significant time on stream (TOS) of 200 hours, the propylene oxide selectivities (relastive to hydrogen peroxide) of the moldings according to the inventive Example 2.2 were compared with the respective moldings according to the prior art (Comparative Examples 1 and 2). For a complete picture, a further comparative testing was carried out (Comparative Example 3) according to which moldings comprising a (non-hollow) TS-1 zeolitic material were subjected to the very same continuous epoxidation reaction conditions according to Reference Example 11. The following results according to Table 3 were obtained:

TABLE 3
Results for catalytic testing according
to Reference Example 11 at a TOS of 200 h

| | propylene oxide | propylene oxide | |
|---|---|---|---|
| | selectivity | selectivity | hydrogen |
| | relative to | relative to | peroxide |
| Molding | hydrogen peroxide/ | propene/ | conversion/ |
| according to | % | % | % |
| Example 1.2 | 96.5 | 97.0 | 90 ± 2 |
| Comparative | 95.0 | 95.0 | 90 ± 2 |
| Example 1 | | | |
| Comparative | 93.0 | 92.0 | 90 ± 2 |
| Example 2 | | | |
| Comparative | 95.0 | 96.0 | 90 ± 2 |
| Example 3 | | | |

Obviously, the molding according to the present invention exhibits the best selectivity values, both relative to hydrogen peroxide and propene, when compared to the moldings of the prior art comprising HTS-1 zeolite according to the Comparative Examples 1 and 2, and also when compared to a molding comprising a (non-hollow) TS-1 zeolite. In particular, it is noted that these results were obtained based on the moldings according to Example 1.2 which, according to the preliminary PO test results according to Example 3. above, may appear to be somewhat inferior in catalytic activity compared to the inventive moldings according to Example 2.2. All the more the superior characteristics of the inventive moldings are shown.

Yet further, it was found that the moldings of the present invention exhibit an excellent stability with respect to the propylene oxide selectivity. In this regard, the moldings of the present invention (according to Example 1.2) as well as the moldings of Comparative Example 3 were subjected to the continuous epoxidation reaction conditions according to Reference Example 11 for a TOS of over 750 hours, and it was found that the high selectivities relative to hydrogen peroxide and propene even show the tendency to increase over time. And it was further found that the highly advantageous characteristics are not only observed for the valuable product of the epoxidation reaction (propylene oxide); at the same time, the moldings of the present invention exhibit significantly lower selectivities with regard to undesired by-products of the epoxidation reaction such as oxygen, hydroperoxides, and methoxypropanols. The respective results are shown in FIGS. 1 and 2, values extracted from the Figures are shown in the Table 4 below:

TABLE 4
Selectivities (propylene oxide, by-products,
secondary products) according to the catalytic
testing according to Reference Example 11

| | | | Comparative |
|---|---|---|---|
| | | Example 1.2 | Example 3 |
| TOS/ | selectivity of # | selectivities/% | |
| h | (relative to H₂O₂) | at a H₂O₂ conversion of 90 ± 2% | |
| 200 | propylene oxide | 96.5 | 95 |
| | oxygen | 0.8 | 0.8 |
| | hydroperoxides | 1.1 | 4.0 |
| | methoxypropanols | 2.1 | 2.1 |
| 300 | propylene oxide | 97 | 95.5 |
| | oxygen | 0.8 | 0.8 |
| | hydroperoxides | 1.35 | 3.8 |
| | methoxypropanols | 1.9 | 2.1 |
| 400 | propylene oxide | 97 | 96 |
| | oxygen | 0.7 | 0.5 |
| | hydroperoxides | 1.5 | 3.2 |
| | methoxypropanols | 1.8 | 1.8 |
| 500 | propylene oxide | 97.5 | 95.5 |
| | oxygen | 0.7 | 1.0 |
| | hydroperoxides | 1.2 | 3.5 |
| | methoxypropanols | 1.5 | 2.0 |

Obviously, the inventive moldings show highly advantageous improved lifetime characteristics in a continuous epoxidation reaction, wherein this continuous mode is the standard mode for industrial-scale epoxidation processes.

### Example A: Preparation of a P-Treated HTS-1

20 g of a commercially available HTS-1 powder (Titanium Silicalite RT-03 of Zhejiang TWRD New Material Co., Ltd., CN) were provided in a vessel. To the zeolitic material, 2.31 g ortho-phosphor acid (aqueous solution, 85 weight-% H₃PO₄) were added and homogenized. The re-suiting mixture was dried in air at a temperature of 110° C. for 12 h. The obtained dried solid material as sieved (mesh size 1.6 mm), and the resulting material was calcined at 500° C. for 5 h in air at heating ramp of 2 K/min. The yield was 20.9 g (split: 2.8 g; undersize particles: 18.1 g).

The resulting material had a TOC of less than 0.1 g/100 g, a Si content of 42 g/100 g, and a Ti content of 1.4 g/100 g, and a P content of 2.7 g/100 g.

### BRIEF DESCRIPTION OF FIGURES

FIG. 1: shows the catalytic performance of the moldings of the present invention for according to Example 1.2 (solid lines) relative to the moldings of Comparative Example 3 (dotted lines), in particular the propylene selectivities relative to hydrogen peroxide (dark grey) and relative to propene (light grey). The lower solid black line shows the hydrogen peroxide conversion, the upper solid black line shows the temperature of the cooling medium flowing through the jacket of the reactor.

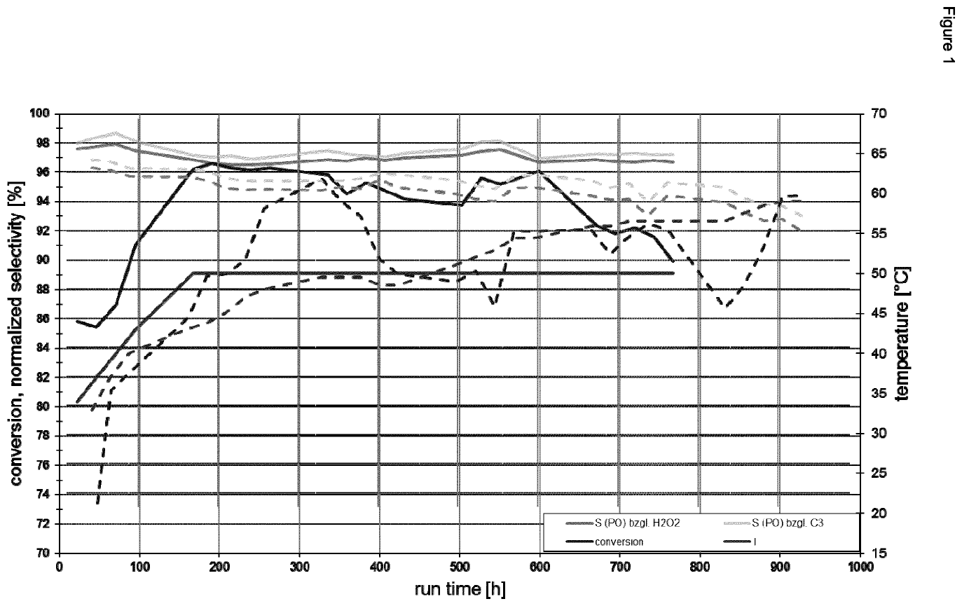

FIG. 2: shows the catalytic performance of the moldings of the present invention for according to Example 1.2 (solid lines) relative to the moldings of Comparative Example 3 (dotted lines), in particular the selectivities of oxygen, hydroxyacetone, hydroperoxides, 1-methoxy-2-propanol and 2-methoxy-1-propanol.

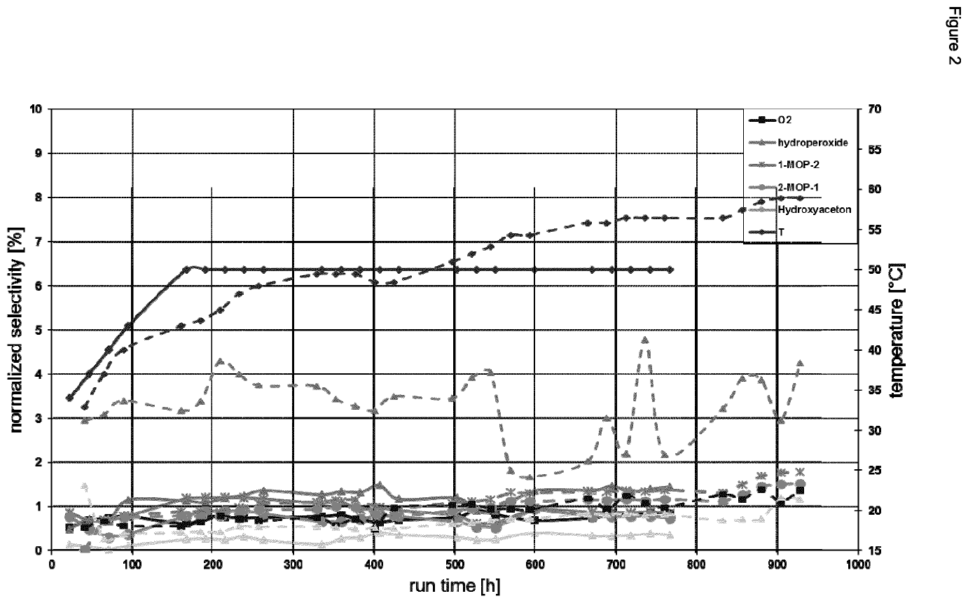

### CITED LITERATURE

M. Liu et al.: “Green and efficient preparation of hollow titanium silicalite-1 by using recycled mother liquid” in Chemical Engineering Journal 2018, vol. 331, p. 194-202
J. Xu et al.: “Effect of triethylamine treatment of titanium silicalite-1 on propylene epoxidation” in Frontiers of Chemical Science and Engineering 2014, vol. 8(4), p. 478-487
M. Liu et al. “Highly Selective Epoxidation of Propylene in a Low-Pressure Continuous Slurry Reactor and the Regeneration of Catalyst” in Industrial+Engineering Chemistry Research 2015, vol. 54(20), p. 5416-5426
CN 108250161 A
CN 103708493 A

1.-15. (canceled)

16. A molding, comprising a zeolitic material having framework type MFI wherein from 98 to 100 weight-% of the zeolitic material consist of Ti, Si, O, and H, and wherein the zeolitic material having framework type MFI exhibits a type IV nitrogen adsorption/desorption, the molding further comprising a silica binder, wherein the molding has a pore volume of at least 0.8 mL/g determined by intrusion mercury porosimetry.

17. The molding of claim 16, wherein the zeolitic material having framework type MFI comprises hollow cavities having a diameter of greater than 5.5 Angstrom, determined via TEM.

18. The molding of claim 16, wherein from 99 to 100 weight % of the zeolitic material having framework type MFI consist of Ti, Si, O, and H.

19. The molding of claim 16, wherein from 99.9 to 100 weight %, of the zeolitic material having framework type MFI consist of Ti, Si, O, and H.

20. The molding of claim 16, wherein the zeolitic material having framework type MFI has a Ti content in the range of from 1.3 to 2.1 weight %, calculated as elemental Ti and based on the weight of zeolitic material.

21. The molding of claim 16, wherein the pore volume is in the range of from 0.8 to 1.5 mL/g,

22. the molding of claim 16, wherein the pore volume is in the range of from 1.0 to 1.3 mL/g.

23. The molding of claim 16, wherein in the molding, the weight ratio of the zeolitic material having framework type MFI relative to the silica binder calculated as SiO₂, MFI:SiO₂, is in the range of from 1:1 to 5:1,

24. The molding of claim 16, wherein in the molding, the weight ratio of the zeolitic material having framework type MFI relative to the silica binder calculated as SiO₂, MFI:SiO₂, is in the range of from 2:1 to 3:1.

25. The molding of any one of claim 16, wherein from 99 to 100 weight %, of the molding consist of the zeolitic material having framework type MFI and the silica binder.

26. A process for preparing a molding comprising a zeolitic material having framework type MFI and a silica binder, the molding according to claim 16, the process comprising
(i) providing a mixture comprising a silica binder precursor and a zeolitic material having framework type MFI, wherein from 98 to 100 weight % of the zeolitic material consist of Ti, Si, O, and H, and wherein the zeolitic material having framework type MFI exhibits a type IV nitrogen adsorption/desorption isotherm;
(ii) shaping the mixture obtained from (i), obtaining a precursor of the molding;
(iii) preparing a mixture comprising the precursor of the molding obtained from (ii) and water, and subjecting the mixture to a water treatment under hydrothermal conditions, obtaining a water-treated precursor of the molding;
(iv) calcining the water-treated precursor of the molding in a gas atmosphere, obtaining the molding.

27. The process of claim 26, wherein the silica binder precursor is selected from the group consisting of a silica sol, a colloidal silica, a wet process silica, a dry process silica, and a mixture of two or more thereof.

28. The process of claim 26, wherein the mixture prepared according to (i) further comprises one or more viscosity modifying and/or mesopore forming agents, wherein the one or more agents are selected from the group consisting of water, alcohols, organic polymers, and mixtures of two or more thereof, wherein the organic polymers are selected from the group consisting of cellulose derivatives, polyalkylene oxides, polystyrenes, and mixtures of two or more thereof.

29. The process of claim 26, wherein in (ii), the mixture is shaped to a strand, the strand having a circular cross-section, wherein in (ii), shaping comprises extruding the mixture.

30. The process of claim 26, wherein shaping according to (ii) further comprises drying the precursor of the molding in a gas atmosphere and calcining the dried precursor of the molding in a gas atmosphere, at a temperature of the gas atmosphere in the range of from 450 to 530° C.

31. The process of claim 26, wherein shaping according to (ii) further comprises drying the precursor of the molding in a gas atmosphere and calcining the dried precursor of the molding in a gas atmosphere, at a temperature of the gas atmosphere in the range of from 480 to 500° C.

32. The process of claim 26, wherein the water treatment according to (iii) comprises a temperature of the mixture in the range of from 100 to 200° C. wherein the water treatment according to (iii) is carried out under autogenous pressure.

33. The process of claim 26, wherein the water treatment according to (iii) comprises a temperature of the mixture in the range of from 140 to 150° C., wherein the water treatment according to (iii) is carried out under autogenous pressure.

34. An absorbent, adsorbent, catalyst, or catalyst component comprising the composition according to claim 16.

35. The catalyst or catalyst component of claim 34, wherein the catalyst or catalyst component is a Lewis acid catalyst or a Lewis acid catalyst component, an isomerization catalyst or an isomerization catalyst component, an oxidation catalyst or an oxidation catalyst component, an aldol condensation catalyst or an aldol condensation catalyst component, or a Prins reaction catalyst or a Prins reaction catalyst component.
